(19) United States
(12) Patent Application Publication
(10) Pub. No.: US 2019/0110969 A1
(43) Pub. Date: Apr. 18, 2019

(54) HAIR GROWTH COMPOSITIONS AND METHODS OF USE

(71) Applicant: North Star Scientific, Inc., Houston, TX (US)

(72) Inventors: Ahmet Korkmaz, Houston, TX (US); Secil Aydinoz, Houston, TX (US)

(73) Assignee: North Star Scientific, Inc., Houston, TX (US)

(21) Appl. No.: 15/959,059

(22) Filed: Apr. 20, 2018

Related U.S. Application Data
(60) Provisional application No. 62/573,643, filed on Oct. 17, 2017.

(51) Int. Cl. A61K 8/35; A61K 8/49; A61K 8/63; A61K 8/92; A61Q 7/00

(52) U.S. Cl. CPC A61K 8/355; A61K 8/492; A61Q 7/00; A61K 8/4953; A61K 8/922; A61K 8/63

(57) ABSTRACT

The present technology provides compositions that include a coenzyme, an epigenetic modifier, a blood circulator, a 5-alpha reductase modulator, and a cosmetically acceptable carrier, as well as methods of preparing and using such compositions. The present technology also provides methods of growing hair and/or stimulating hair growth where the method includes administering a cosmetically effective amount of a coenzyme and a cosmetically effective amount of an epigenetic modifier.

### CROSS-REFERENCE TO RELATED APPLICATIONS

This application claims the benefit of U.S. Provisional Patent Application No. 62/573,643, filed on Oct. 17, 2017, the entire disclosure of which is incorporated herein by reference in its entirety for any and all purposes.

### BACKGROUND

Society places considerable emphasis on external appearance, of which, hair is an important component. Scientists and medical doctors claim that hair provides the most important contribution to the overall appearance of the human body. Hair loss, although a natural part of the aging process, is often a cause for concern. The profound symbolic and psychosocial importance attached to hair is reflected by the anxiety and distress resulting from its loss.

Several research groups have assessed the psychological effects of hair loss on women by comparing them with balding men and female control subjects. The research groups confirmed the potentially adverse psychosocial effect of this common dermatological condition. Hair loss and thinning continue to remain common dermatological complaints despite the introduction of many treatment alternatives.

In particular, androgenetic alopecia is the most common hair loss disorder, affecting both men and women. Initial signs of androgenetic alopecia usually develop during teenage years leading to progressive hair loss with a pattern distribution. Moreover, its frequency increases with age and affects up to 80% Caucasian men and 42% of women. Subjects diagnosed with androgenetic alopecia may undergo significant impairment of quality of life. Despite the high prevalence and the variety of therapeutic options available, there have been no national or international evidence-based guidelines for the treatment of androgenetic alopecia in men and women thus far.

Available treatments for alopecia are limited in number as well as in their efficacy. For example, minoxidil and finasteride have positive but limited effects on hair loss. Thus, there is a need for an effective cosmetic for alopecia.

### SUMMARY

In one aspect, provided herein is a composition comprising: a) a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof; b) a cosmetically effective amount of one or more of an epigenetic modifier; c) a cosmetically effective amount of a blood circulator; d) a cosmetically effective amount of a 5-alpha reductase modulator (such as a 5-alpha reductase inhibitor, a 5-alpha reductase receptor modifier, and/or a 5-alpha reductase receptor blocker); e) a cosmetically acceptable carrier; and f) an antioxidant.

In a related aspect, a method is provided herein of growing hair and/or stimulating hair growth comprising administering to a subject in need thereof a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof; and a cosmetically effective amount of an epigenetic modifier. The method may include administering a composition of any embodiment described herein. In any embodiment herein, the hair may include one or more of an eyebrow hair, an eyelash hair, a mustache hair, and a beard hair.

### BRIEF DESCRIPTION OF THE DRAWINGS

FIG. 1 is a before (left panel) and after (right panel) picture of the hair status of a male subject who was administered a composition provided herein twice a day for 45 days.

FIGS. 2A-B are a before (FIG. 2A) and after (FIG. 2B) picture of the hair status of a male subject who was administered a composition provided herein twice a day for 45 days.

FIGS. 3A-B are a before (FIG. 3A) and after (FIG. 3B) picture of the hair status of a male subject who was administered a composition provided herein twice a day for 45 days.

FIGS. 4A-B are a before (FIG. 4A) and after (FIG. 4B) picture of the hair status of a male subject who was administered a composition provided herein twice a day for 90 days.

FIGS. 5A-B are a before (FIG. 5A) and after (FIG. 5B) picture of the hair status of a male subject who was administered a composition provided herein twice a day for 30 days.

FIGS. 6A-B are a before (FIG. 6A) and after (FIG. 6B) picture of the hair status of a male subject who was administered a composition provided herein twice a day for 120 days.

FIGS. 7A-B are a before (FIG. 7A) and after (FIG. 7B) picture of the hair status of a male subject who was administered a composition provided herein twice a day for 84 days.

FIGS. 8A-C are a before (FIG. 8A) and after (FIGS. 8B-8C) pictures of the hair status of a male subject who was administered a composition provided herein twice a day, where FIG. 8B shows the hair status after 90 days and FIG. 8C the hair status after 6 months.

FIGS. 9A-B are a before (FIG. 9A) and after (FIG. 9B) picture of the hair status of a male subject who was administered a composition provided herein twice a day for 3 months.

FIGS. 10A-B are a before (FIG. 10A) and after (FIG. 10B) picture of the hair status of a male subject who was administered a composition provided herein twice a day for 3 months.

FIGS. 11A-B are a before (FIG. 11A) and after (FIG. 11B) picture of the hair status of a male subject who was administered a composition provided herein twice a day for 3 months.

FIGS. 12A-B are a before (FIG. 12A) and after (FIG. 12B) picture of the hair status of a male subject who was administered a composition provided herein twice a day for 3 months.

FIGS. 13A-B are a before (FIG. 13A) and after (FIG. 13B) picture of the hair status of a male subject who was administered a composition provided herein twice a day for 3 months.

FIGS. 14A-B are a before (FIG. 14A) and after (FIG. 14B) picture of the hair status of a male subject who was administered a composition provided herein twice a day for 3 months.

FIGS. 15A-B are a before (FIG. 15A) and after (FIG. 15B) picture of the hair status of a male subject who was administered a composition provided herein twice a day for 3 months.

### DETAILED DESCRIPTION

All publications cited herein, including internet articles, books, handbooks, journal articles, patents and patent applications, whether supra or infra, are hereby incorporated by reference in their entirety.

Before describing the present disclosure in detail, it is to be understood that this disclosure is not limited to particular administration modes, subject populations, and the like, as such may vary, as will be apparent from the accompanying description and figures.

Technical and scientific terms used herein have the meanings commonly understood by persons of ordinary skill in the art to which the present disclosure pertains, unless otherwise defined. Reference is made herein to various methodologies known to persons of ordinary skill in the art. Suitable materials and/or methods known to persons of ordinary skill in the art can be utilized in carrying out the present disclosure. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only. Thus, for example, reference to “a drug” includes a single drug as well as two or more of the same or different drugs, reference to “an optional excipient” refers to a single optional excipient as well as two or more of the same or different optional excipients, and the like.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number, which, in the context in which it is presented, provides the substantial equivalent of the specifically recited number.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure. When an embodiment is defined by one of these terms (e.g., “comprising”) it should be understood that this disclosure also includes alternative embodiments, such as “consisting essentially of” and “consisting of” for said embodiment.

As used herein, the terms “subject”, “individual” or “patient” are used interchangeably herein and refer to a vertebrate. In some embodiments, the vertebrate is preferably a mammal, which includes, but is not limited to, mice, rodents, rats, simians, humans, farm animals, dogs, cats, sport animals, and pets.

As used herein, the terms “administer,” “administration,” or “administering” refer to (1) providing, giving, dosing and/or prescribing, such as by either a health professional or his or her authorized agent or under his direction, (2) putting into, such as by a health professional or his or her authorized agent or under his direction, and (3) taking or consuming, such as by the subject.

As used herein, the terms “treat”, “treating” or “treatment” include alleviating, abating or ameliorating a disease or condition (e.g., cosmetic condition) or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any cosmetic and/or prophylactic benefit.

As used herein, the term “cosmetically effective amount” refers to a dose that provides the specific cosmetic and/or pharmacological effect for which the drug is administered in a subject in need of such cosmetic treatment. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein. A cosmetically effective amount will not always be effective in treating the conditions described herein, even though such dose is deemed to be a cosmetically effective amount by those of skill in the art. For convenience only, exemplary doses and cosmetically effective amounts are provided below with reference to adult human subjects. Those skilled in the art can adjust such amounts in accordance with standard practices as needed to treat a specific subject and/or condition/disease.

“Cosmetically acceptable excipient or carrier” refers to an excipient that may optionally be included in the compositions of the disclosure and that causes no significant adverse toxicological effects to the subject.

Cosmetically acceptable salts of compounds and components described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, cosmetically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). In addition to the commercial availability of many such salts, such salts can be prepared in situ during isolation and purification of the compounds or by separately reacting the purified compound in its free base or free acid form with a suitable acid or base, respectively, and isolating the salt thus formed.

The compounds and components of the present technology may exist as/be commercially available as/used as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

“Active molecule” or “active agent” as described herein includes any agent, drug, compound, composition of matter or mixture which provides some cosmetic, often beneficial, effect that can be demonstrated in-vivo or in vitro. This includes foods, food supplements, nutrients, nutraceuticals, drugs, vaccines, antibodies, vitamins, and other beneficial agents. As used herein, the terms further include any physiologically, cosmetically, and/or pharmacologically active substance that produces a localized or systemic effect in a subject. In specific embodiments, the active molecule or active agent may include ubiquinone or a cosmetically acceptable salt thereof. In specific embodiments, the active molecule or active agent may include an epigenetic modifier.

As used herein, the term “epigenetic modifier” refers to an agent that has the ability to regulate, modify, and/or reverse an epigenetic modification of a nucleic acid, such as DNA or RNA, or a protein, such as a histone. Non-limiting illustrative examples of an epigenetic modifier include melatonin, epigallocatechine gallate (EGCG), resveratrol, curcumin, valproic acid, azacytidine, decitabine, and zebularine. In particular, melatonin is an epigenetic modifier & epigenetic modulator.

As used herein, the terms “terminal antioxidant” and “suicidal antioxidant” are interchangeable and refer to an antioxidant that cannot be oxidized either in a mitochondrion or in a mammalian body.

As used herein, the terms “non-terminal antioxidant” and “non-suicidal antioxidant” are interchangeable and refer to an antioxidant that can be oxidized either in a mitochondrion or in a mammalian body.

“Substantially” or “essentially” means nearly totally or completely, for instance, 95%, 96%, 97%, 98%, 99%, or greater of some given quantity.

“Optional” or “optionally” means that the subsequently described circumstance may or may not occur, so that the description includes instances where the circumstance occurs and instances where it does not.

It is to be understood that a “volume percent” or “% by volume” of a component in a composition is determined at room temperature (about 23° C.) based on the sum of the initial volumes of each individual component, not the final volume of combined components. However, for those components that are solid at room temperature (about 23° C.), it is to be understood in these instances that the “weight percent” or “% by weight” is intended based on the sum of the initial weights of each individual component.

Other objects, features, and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### Compositions

In one aspect, provided herein is a composition comprising: a) a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof; b) a cosmetically effective amount of one or more of an epigenetic modifier; c) a cosmetically effective amount of a blood circulator; d) a cosmetically effective amount of a 5-alpha reductase modulator (such as a 5-alpha reductase inhibitor, a 5-alpha reductase receptor modifier, and/or a 5-alpha reductase receptor blocker); and e) a cosmetically acceptable carrier f) and optionally one or more antioxidants.

In some embodiments, the composition comprises about 0.1-1.5%, about 0.3-1.0%, 0.5-1.0%, about 0.3-0.8%, or about 0.4-0.6% by volume of coenzyme. In some embodiments, the composition comprises about 0.5-1.0% by volume of coenzyme. In some embodiments, the composition comprises about 0.4-0.6% by volume of coenzyme. In some embodiments, the composition comprises about 0.1%, about 0.25%, about 0.3%, about 0.4%, about 0.5%, about 0.6%, about 0.7%, about 0.75%, about 0.8%, about 0.9%, about 1.0%, about 1.1%, about 1.2%, about 1.3%, about 1.4%, about 1.5% (or any range including and/or in between any two of these values) by volume of coenzyme. In some embodiments, the composition comprises about 0.4% by volume of coenzyme. In some embodiments, the composition comprises about 0.5% by volume of coenzyme. In some embodiments, the composition comprises about 0.6% by volume of coenzyme.

In some embodiments, the composition comprises about 0.1-1.5%, about 0.3-1.0%, 0.5-1.0%, about 0.3-0.8%, or about 0.4-0.6% by volume of ubiquinone. In some embodiments, the composition comprises about 0.5-1.0% by volume of ubiquinone. In some embodiments, the composition comprises about 0.4-0.6% by volume of ubiquinone. In some embodiments, the composition comprises about 0.1%, about 0.25%, about 0.3%, about 0.4%, about 0.5%, about 0.6%, about 0.7%, about 0.75%, about 0.8%, about 0.9%, about 1.0%, about 1.1%, about 1.2%, about 1.3%, about 1.4%, about 1.5% (or any range including and/or in between any two of these values) by volume of ubiquinone. In some embodiments, the composition comprises about 0.4% by volume of ubiquinone. In some embodiments, the composition comprises about 0.5% by volume of ubiquinone. In some embodiments, the composition comprises about 0.6% by volume of ubiquinone.

In some embodiments, the epigenetic modifier reverses an epigenetic modification. In some embodiments, the epigenetic modifier modifies one or more of dermal papilla cells, melanocytes, or keratinocytes. Non-limiting illustrative examples of an epigenetic modifier include melatonin, epigallocatechine gallate (EGCG), valproic acid, azacytidine, decitabine, zebularine. In some embodiments, the epigenetic modifier comprises one or more of melatonin, EGCG, valproic acid, azacytidine, decitabine, zebularine. In some embodiments, the composition comprises about 0.1-2.5%, about 0.5-2.5%, about 0.5-2.0%, about 0.5-1.5%, about 0.5-1.0%, about 1.5-2.0%, or about 0.1-0.5% by volume of an epigenetic modifier. In some embodiments, the composition comprises about 0.1%, about 0.25%, about 0.3%, about 0.5%, about 0.7%, about 0.75%, about 0.8%, about 1.0%, about 1.25%, about 1.5%, about 1.75%, about 2.0%, about 2.25%, or about 2.5% (or any range including and/or in between any two of these values) by volume of the epigenetic modifier.

In some embodiments, the epigenetic modifier comprises melatonin. In some embodiments, the composition comprises about 0.5-2.0%, about 1.0-1.5%, or about 1.0-2.0% by volume of melatonin. In some embodiments, the composition comprises about 0.25%, about 0.5%, about 0.75%, about 1.0%, about 1.25%, about 1.5%, about 1.75%, about 2.0%, about 2.25%, about 2.5% (or any range including and/or in between any two of these values) by volume of melatonin. In some embodiments, the composition comprises about 1.0% by volume of melatonin. In some embodiments, the composition comprises about 1.5% by volume of melatonin.

In some embodiments, the epigenetic modifier comprises EGCG. In some embodiments, the composition comprises about 0.1-2.5%, about 0.5-1.5%, or about 0.1-0.5% by volume of EGCG. In some embodiments, the composition comprises about 0.1%, 0.3%, 0.5%, 0.7%, or about 0.8% (or any range including and/or in between any two of these values) by volume of EGCG. In some embodiments, the composition comprises about 0.5% by volume of EGCG.

In some embodiments, the epigenetic modifier comprises valproic acid (and/or a cosmetically acceptable salt thereof). In some embodiments, the composition comprises about 0.1-2.5%, about 0.5-1.5%, or about 0.1-0.5% by volume of valproic acid. In some embodiments, the composition comprises about 0.1%, 0.3%, 0.5%, 0.7%, or about 0.8% (or any range including and/or in between any two of these values) by volume of valproic acid. In some embodiments, the composition comprises about 0.5% by volume of valproic acid.

In some embodiments, the epigenetic modifier comprises azacytidine. In some embodiments, the composition comprises about 0.1-2.5%, about 0.5-1.5%, or about 0.1-0.5% by volume of azacytidine. In some embodiments, the composition comprises about 0.1%, 0.3%, 0.5%, 0.7%, or about 0.8% (or any range including and/or in between any two of these values) by volume of azacytidine. In some embodiments, the composition comprises about 0.5% by volume of azacytidine.

In some embodiments, the epigenetic modifier comprises decitabine. In some embodiments, the composition comprises about 0.1-2.5%, about 0.5-1.5%, or about 0.1-0.5% by volume of decitabine. In some embodiments, the composition comprises about 0.1%, 0.3%, 0.5%, 0.7%, or about 0.8% (or any range including and/or in between any two of these values) by volume of decitabine. In some embodiments, the composition comprises about 0.5% by volume of decitabine.

In some embodiments, the epigenetic modifier comprises zebularine. In some embodiments, the composition comprises about 0.1-2.5%, about 0.5-1.5%, or about 0.1-0.5% by volume of zebularine. In some embodiments, the composition comprises about 0.1%, 0.3%, 0.5%, 0.7%, or about 0.8% (or any range including and/or in between any two of these values) by volume of zebularine. In some embodiments, the composition comprises about 0.5% by volume of zebularine.

In some embodiments, the epigenetic modifier comprises any combination of two or more of the group of melatonin, EGCG, valproic acid, azacytidine, decitabine, and zebularine.

In some embodiments, the epigenetic modifier comprises melatonin and EGCG. In some embodiments, the composition comprises about 1.0-2.5% of melatonin and EGCG. In some embodiments, the composition comprises about 1.0%, about 1.5%, about 2.0%, or about 2.5% (or any range including and/or in between any two of these values) by volume of melatonin and EGCG. In some embodiments, the composition comprises about 1.5% or 2.0% by volume of EGCG.

In some embodiments, the composition provided herein further comprises a terminal antioxidant or suicidal antioxidant. Non-limiting illustrative examples of a terminal antioxidant or suicidal antioxidant include melatonin, 5-methoxytryptamine, cyclic 3-hydroxymelatonin, and N-acetyl-5-methoxy kynuramine. In some embodiments, the terminal or suicidal antioxidants comprise one or more of the group of melatonin, 5-methoxytryptamine, cyclic 3-hydroxymelatonin, and N-acetyl-5-methoxy kynuramine. In some embodiments, the composition comprises about 0.5-2.0% or about 1.0-1.5% by volume of the terminal antioxidant or suicidal antioxidant. In some embodiments, the composition comprises about 0.5%, about 1.0%, or about 1.5% (or any range including and/or in between any two of these values) by volume of the terminal antioxidant or suicidal antioxidant.

In some embodiments, the terminal antioxidants or suicidal antioxidants comprise melatonin. In some embodiments, the composition comprises about 0.5-2.0%, about 1.0-1.5%, or about 1.0-2.0% by volume of melatonin. In some embodiments, the composition comprises about 0.25%, about 0.5%, about 0.75%, about 1.0%, about 1.25%, about 1.5%, about 1.75%, about 2.0%, about 2.25%, or about 2.5% (or any range including and/or in between any two of these values) by volume of melatonin. In some embodiments, the composition comprises about 1.0% by volume of melatonin. In some embodiments, the composition comprises about 1.5% by volume of melatonin.

In some embodiments, the terminal antioxidants or suicidal antioxidants comprise 5-methoxytryptamine. In some embodiments, the composition comprises about 0.5-2.0%, about 1.0-1.5%, or about 1.0-2.0% by volume of 5-methoxytryptamine. In some embodiments, the composition comprises about 0.25%, about 0.5%, about 0.75%, about 1.0%, about 1.25%, about 1.5%, about 1.75%, about 2.0%, about 2.25%, or about 2.5% (or any range including and/or in between any two of these values) by volume of 5-methoxytryptamine. In some embodiments, the composition comprises about 1.0% by volume of 5-methoxytryptamine. In some embodiments, the composition comprises about 1.5% by volume of 5-methoxytryptamine.

In some embodiments, the terminal or suicidal antioxidants comprises cyclic 3-hydroxymelatonin. In some embodiments, the composition comprises about 0.5-2.0%, about 1.0-1.5%, or about 1.0-2.0% by volume of cyclic 3-hydroxymelatonin. In some embodiments, the composition comprises about 0.5%, 1.0%, 1.5%, or 2.0% (or any range including and/or in between any two of these values) by volume of cyclic 3-hydroxymelatonin. In some embodiments, the composition comprises about 1.5% by volume of cyclic 3-hydroxymelatonin. In some embodiments, the composition comprises about 1.0% by volume of cyclic 3-hydroxymelatonin.

In some embodiments, the terminal or suicidal antioxidants comprises N-acetyl-5-methoxy kynuramine. In some embodiments, the composition comprises about 0.5-2.0% or about 1.0-1.5% by volume of N-acetyl-5-methoxy kynuramine. In some embodiments, the composition comprises about 0.25%, about 0.5%, about 0.75%, about 1.0%, about 1.25%, about 1.5%, about 1.75%, about 2.0%, about 2.25%, or about 2.5% (or any range including and/or in between any two of these values) by volume of N-acetyl-5-methoxy kynuramine. In some embodiments, the composition comprises about 1.0% by volume of N-acetyl-5-methoxy kynuramine. In some embodiments, the composition comprises about 1.5% by volume of N-acetyl-5-methoxy kynuramine.

In some embodiments, the composition provided herein further comprises a moisturizer. Non-limiting illustrative examples of a moisturizer include urea and dexpanthenol (vitamin B5). In some embodiments, the moisturizer comprises urea, dexpanthenol, or both. In some embodiments, the composition comprises about 0.25-1.0% by volume of the moisturizer. In some embodiments, the composition comprises about 0.25%, about 0.5%, about 0.75%, or about 1.0% (or any range including and/or in between any two of these values) by volume of the moisturizer.

In some embodiments, the moisturizer comprises urea. In some embodiments, the composition comprises about 0.25-1.0% by volume of urea. In some embodiments, the composition comprises about 0.25%, about 0.5%, about 0.75%, or about 1.0% (or any range including and/or in between any two of these values) by volume of urea. In some embodiments, the composition comprises about 0.25% by volume of urea. In some embodiments, the composition comprises about 0.5% by volume of urea.

In some embodiments, the moisturizer comprises dexpanthenol. In some embodiments, the composition comprises about 0.25-1.0% by volume of dexpanthenol. In some embodiments, the composition comprises about 0.25%, about 0.5%, about 0.75%, or about 1.0% (or any range including and/or in between any two of these values) by volume of dexpanthenol. In some embodiments, the composition comprises about 0.5% by volume of dexpanthenol.

In some embodiments, the moisturizer comprises urea and dexpanthenol.

In some embodiments, the composition provided herein further comprises an anti-fungal agent. Non-limiting illustrative examples of an anti-fungal agent include allicin and eugenol. In some embodiments, the anti-fungal agent comprises allicin, eugenol, or both. In some embodiments, the composition comprises about 0.05-2.5%, about 0.1-2.0%, about 0.1-1.75%, about 0.2-1.75%, about 0.5-2.5%, about 0.5-2.0%, about 0.5-1.75%, about 0.05-0.3%, about 0.05-0.2%, or about 0.07-0.15% by volume of the anti-fungal agent. In some embodiments, the composition comprises about 0.05%, about 0.06%, about 0.07%, about 0.08%, about 0.09%, about 0.1%, about 0.15%, about 0.2%, about 0.25%, about 0.3%, about 0.4%, about 0.5%, about 0.6%, about 0.7%, about 0.75%, about 0.8%, about 0.9%, about 1.0%, about 1.2%, about 1.25%, about 1.3%, about 1.4%, about 1.5%, about 1.6%, about 1.7%, about 1.75%, about 1.8%, about 2.0%, about 2.1%, about 2.25%, about 2.3%, about 2.4%, or about 2.5% (or any range including and/or in between any two of these values) by volume of the anti-fungal agent.

In some embodiments, the anti-fungal agent comprises allicin. In some embodiments, the composition comprises about 0.05-0.2% by volume of allicin. In some embodiments, the composition comprises about 0.07-0.15% by volume of allicin. In some embodiments, the composition comprises about 0.05%, about 0.06%, about 0.07%, about 0.08%, about 0.09%, or about 0.1% (or any range including and/or in between any two of these values) by volume of allicin. In some embodiments, the composition comprises about 0.1% by volume of allicin.

In some embodiments, the anti-fungal agent comprises eugenol. In some embodiments, the composition comprises about 0.1-1.75%, about 0.5-1.75%, about 0.5-2.5%, or about 0.5-2.0%, by volume of eugenol. In some embodiments, the composition comprises about 0.1-1.75% by volume of eugenol. In some embodiments, the composition comprises about 0.05-0.2% by volume of eugenol. In some embodiments, the composition comprises about 0.07-0.15% by volume of eugenol. In some embodiments, the composition comprises about 0.05%, about 0.06%, about 0.07%, about 0.08%, about 0.09%, about 0.1%, about 0.25%, about 0.5%, about 0.75%, about 1.0%, about 1.25%, about 1.5%, about 1.75%, or about 2.0% (or any range including and/or in between any two of these values) by volume of eugenol. In some embodiments, the composition comprises about 0.1% by volume of eugenol.

In some embodiments, the anti-fungal agent comprises allicin and eugenol. In some embodiments, the composition comprises about 0.05-2.5% or about 0.2-1.75% by volume of allicin and eugenol. In some embodiments, the composition comprises about 0.2%, about 0.3%, about 0.4%, about 0.5%, about 0.6%, about 0.7%, about 0.8%, about 0.9%, about 1.0%, about 1.2%, about 1.3%, about 1.4%, about 1.5%, about 1.6%, about 1.7%, about 1.8%, about 2.0%, about 2.1%, about 2.3%, about 2.4%, or about 2.5% (or any range including and/or in between any two of these values) by volume of allicin and eugenol. In some embodiments, the composition comprises about 0.2% by volume of allicin and eugenol.

In some embodiments, the composition provided herein further comprises an ultra-violet light filter (UV filter). Non-limiting illustrative examples of a UV filter include zinc oxide, allicin, and beta-carotene. In some embodiments, the UV filter comprises one or more of the group of zinc oxide, allicin, and beta-carotene. In some embodiments, the composition comprises 0.01-0.7%, about 0.01-0.05%, about 0.05-0.7%, about 0.05-0.5%, about 0.05-0.4%, about 0.05-0.2%, about 0.07-0.15%, or about 0.1-0.3% by volume of the UV filter. In some embodiments, the composition comprises about 0.05%, about 0.06%, about 0.07%, about 0.08%, about 0.09%, about 0.1%, about 0.2%, about 0.25%, about 0.3%, about 0.35%, about 0.4%, about 0.5%, about 0.6%, or about 0.7% (or any range including and/or in between any two of these values) by volume of the UV filter.

In some embodiments, the UV filter comprises zinc oxide. In some embodiments, the composition comprises about 0.1-0.3% by volume of zinc oxide. In some embodiments, the composition comprises about 0.1%, about 0.2%, about 0.25%, or about 0.3% (or any range including and/or in between any two of these values) by volume of zinc oxide. In some embodiments, the composition comprises about 0.25% of zinc oxide.

In some embodiments, the UV filter comprises allicin. In some embodiments, the composition comprises about 0.05-0.2% by volume of allicin. In some embodiments, the composition comprises about 0.07-0.15% by volume of allicin. In some embodiments, the composition comprises about 0.05%, about 0.06%, about 0.07%, about 0.08%, about 0.09%, or about 0.1% (or any range including and/or in between any two of these values) by volume of allicin. In some embodiments, the composition comprises about 0.1% by volume of allicin.

In some embodiments, the UV filter comprises beta-carotene. In some embodiments, the composition comprises about 0.01-0.05%, about 0.05-0.5%, about 0.05-0.2%, or about 0.1-0.3% by volume of beta-carotene. In some embodiments, the composition comprises about 0.05%, about 0.06%, about 0.07%, about 0.08%, about 0.1%, about 0.2%, or about 0.3% (or any range including and/or in between any two of these values) by volume of beta-carotene. In some embodiments, the composition comprises about 0.05% by volume of beta-carotene.

In some embodiments, the UV filter comprises a mixture of two or more of the group of zinc oxide, allicin, and beta-carotene. In some embodiments, the UV filter comprises zinc oxide, allicin, and beta-carotene.

In some embodiments, the composition provided herein further comprises a piloerector muscle relaxer. Non-limiting illustrative examples of a piloerector muscle relaxer include an alpha-1-receptor blocker, a sympatholithic agent, or a monoclonal antibody. In some embodiments, the piloerector muscle relaxer comprises an alpha-1-receptor blocker. Non-limiting illustrative examples of an alpha-1-receptor blocker include prazosin, hydralazine, tamsulosin, and melatonin. In some embodiments, the alpha-1-receptor blocker comprises prazosin, melatonin, or both.

In some embodiments, the composition comprises about 0.1-2.5%, about 0.5-2.5%, about 0.5-2.0%, about 0.5-1.5%, about 0.5-1.0%, about 1.5-2.0%, or about 0.1-0.5% by volume of the alpha-1-receptor blocker. In some embodiments, the composition comprises about 0.1%, about 0.25%, about 0.3%, about 0.5%, about 0.7%, about 0.75%, about 0.8%, about 1.0%, about 1.25%, about 1.5%, about 1.75%, about 2.0%, about 2.25%, or about 2.5% (or any range including and/or in between any two of these values) by volume of the an alpha-1-receptor blocker.

In some embodiments, the alpha-1-receptor blocker comprises prazosin. In some embodiments, the composition comprises about 0.5-2.0%, about 1.0-1.5%, or about 1.0-2.0% by volume of prazosin. In some embodiments, the composition comprises about 0.25%, about 0.5%, about 0.75%, about 1.0%, about 1.25%, about 1.5%, about 1.75%, about 2.0%, about 2.25%, or about 2.5% (or any range including and/or in between any two of these values) by volume of prazosin. In some embodiments, the composition comprises about 1.0% by volume of prazosin. In some embodiments, the composition comprises about 1.5% by volume of prazosin.

In some embodiments, the alpha-1-receptor blocker comprises melatonin. In some embodiments, the composition comprises about 0.5-2.0%, about 1.0-1.5%, or about 1.0-2.0% by volume of melatonin. In some embodiments, the composition comprises about 0.25%, about 0.5%, about 0.75%, about 1.0%, about 1.25%, about 1.5%, about 1.75%, about 2.0%, about 2.25%, or about 2.5% (or any range including and/or in between any two of these values) by volume of melatonin. In some embodiments, the composition comprises about 1.0% by volume of melatonin. In some embodiments, the composition comprises about 1.5% by volume of melatonin.

In some embodiments, the alpha-1-receptor blocker comprises prazosin and melatonin.

In some embodiments, the composition provided herein further comprises a blood circulator. Blood circulators include, but are not limited to, vasodilators. Non-limiting illustrative examples of a blood circulator include minoxidil, pyrrolidinyl diaminopyrimidine oxide, arginine, lidocaine, capsaicin, and curcumin. In some embodiments, the blood circulator comprises one or more of a member of the group selected from minoxidil, pyrrolidinyl diaminopyrimidine oxide, arginine, lidocaine, capsaicin, and curcumin. In some embodiments, the composition comprises about 0.05-6.0%, about 0.1-6.0%, about 0.5-6.0%, about 0.1-3.0%, about 1.0-5.5%, about 1.5-5.0%, about 0.5-2.5%, about 0.05-0.2%, or about 0.07-0.15% by volume of the blood circulator. The total amount of blood circulator in the composition, by volume, may be about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0% or any range including and/or in between any two of these values. If the composition is intended for a female, the composition may preferably include curcumin as at least one of the blood circulators of the composition. For example, if the composition is intended for a female, the composition may include about 0.05% to about 6.0% by volume of curcumin, such as about 0.5% to about 1.0% by volume of curcumin.

In some embodiments, the blood circulator comprises minoxidil and/or a cosmetically acceptable salt thereof (such as minoxidil hydrochloride) and/or comprises minoxidil-d₁₀ (CAS Number 1020718-66-4) and/or a cosmetically acceptable salt thereof. In some embodiments, the composition comprises about 1.5-5.0% by volume of minoxidil and/or minoxidil-d₁₀ (and/or a cosmetically acceptable salt thereof of either). In some embodiments, the composition comprises about 1.0%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, or about 5.5% (or any range including and/or in between any two of these values) by volume of minoxidil and/or minoxidil-d₁₀ (and/or a cosmetically acceptable salt thereof of either). In some embodiments, the composition comprises about 5.0% by volume of minoxidil.

In some embodiments, the blood circulator comprises pyrrolidinyl diaminopyrimidine oxide. In some embodiments, the composition comprises about 0.1-5.0%, about 0.25-5.0%, about 0.1-3.0%, or about 0.5-2.5% by volume of pyrrolidinyl diaminopyrimidine oxide. In some embodiments, the composition comprises about 0.25%, about 0.5%, about 0.75%, about 1.0%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, or about 5.5% (or any range including and/or in between any two of these values) by volume of pyrrolidinyl diaminopyrimidine oxide. In some embodiments, the composition comprises about 5.0% by volume of pyrrolidinyl diaminopyrimidine oxide.

In some embodiments, the blood circulator comprises arginine. In some embodiments, the composition comprises about 0.05-0.2% by volume of arginine. In some embodiments, the composition comprises about 0.07-0.15% by volume of arginine. In some embodiments, the composition comprises about 0.05%, about 0.06%, about 0.07%, about 0.08%, about 0.09%, or about 0.1% (or any range including and/or in between any two of these values) by volume of arginine. In some embodiments, the composition comprises about 0.1% by volume of arginine.

In some embodiments, the blood circulator comprises lidocaine. In some embodiments, the composition comprises about 0.05-0.2% by volume of lidocaine. In some embodiments, the composition comprises about 0.07-0.15% by volume of lidocaine. In some embodiments, the composition comprises about 0.05%, about 0.06%, about 0.07%, about 0.08%, about 0.09%, or about 0.1% (or any range including and/or in between any two of these values) by volume of lidocaine. In some embodiments, the composition comprises about 0.1% by volume of lidocaine.

In some embodiments, the blood circulator comprises capsaicin. In some embodiments, the composition comprises about 0.05-0.2% by volume of capsaicin. In some embodiments, the composition comprises about 0.07-0.15% by volume of capsaicin. In some embodiments, the composition comprises about 0.05%, about 0.06%, about 0.07%, about 0.08%, about 0.09%, or about 0.1% (or any range including and/or in between any two of these values) by volume of capsaicin. In some embodiments, the composition comprises about 0.1% by volume of capsaicin.

In some embodiments, the blood circulator comprises curcumin. In some embodiments, the composition comprises about 0.05-0.2% by volume of curcumin. In some embodiments, the composition comprises about 0.07-0.15% by volume of curcumin. In some embodiments, the composition comprises about 0.05%, about 0.06%, about 0.07%, about 0.08%, about 0.09%, or about 0.1% (or any range including and/or in between any two of these values) by volume of curcumin. In some embodiments, the composition comprises about 0.1% by volume of curcumin.

In some embodiments, the blood circulator comprises two, three, four, five, or all of a group of minoxidil, pyrrolidinyl diaminopyrimidine oxide, arginine, lidocaine, capsaicin, and curcumin.

In some embodiments, the composition provided herein further comprises a 5-alpha reductase modulator. Non-limiting illustrative examples of a 5-alpha reductase modulator include emodin (as well as its metabolites and derivates such as emodin 8-glucoside and aloe-emodin), finasteride, eugenol, dutasteride, enoxolone, equol (such as (S)-equol,(R)-equol, as well as a racemic mixture of equol), 3-(4-hydroxyphenyl)chroman-4,7-diol, genistein (as well as its derivates and metabolites such as genisitin), sophoricoside, daizein (as well as its metabolites such as daidzin), 1,2-dimyristoyl-sn-glycero-3-phospo-L-serine sodium salt, kaempferol (as well as its methobolites or derivates such as kaempferol, kaempferol 3-glucoside, kaempferol 7-O-β-D-glucopyranoside, kaempferol 3-O-β-rutinoside, kaempferol 3-O-D-galactoside, kaempferol 7-O-neohesperidoside, kaempferol 3-rutinoside 4′-glucoside, kaempferitrin, robinin, afzelin, and leucoside), isorhamnetin (as well as its derivates and metabolites such as isorhammetin-3-O-β-D-glucotrioside and isorhammetin 3-O-glucoside), naringenin (as well as its metabolites and derivates such as naringenin 4′,7-dimethyl ether, naringenin-6-C-glucoside, narirutin, 5-O-methylnaringenin, and prunin), myricetin (as well as its derivates and metabolites such as myricitrin, myricetin 3-O-rutinoside, and myricetin 3-rutinoside-7-glucoside), baicalein (as well as its derivates and metabolites such as baicalin, baicalin hydrate, and biochanin A), glycitein, 5,6,7-dihydroxyflavone, fisetin, caffeic acid phenethyl ester, octyl gallate, lauryl gallate, lignan, enterolactone, kolaflavanone, kolaviron, and secoisolariciresinol diglucoside. In some embodiments, the 5-alpha reductase modulator comprises one or more of the group selected from emodin (as well as its metabolites and derivates such as emodin 8-glucoside and aloe-emodin), finasteride, eugenol, dutasteride, enoxolone, equol (such as (S)-equol,(R)-equol, as well as a racemic mixture of equol), 3-(4-hydroxyphenyl)chroman-4,7-diol, genistein (as well as its derivates and metabolites such as genisitin), sophoricoside, daizein (as well as its metabolites such as daidzin), 1,2-dimyristoyl-sn-glycero-3-phospo-L-serine sodium salt, kaempferol (as well as its methobolites or derivates such as kaempferol, kaempferol 3-glucoside, kaempferol 7-O-β-D-glucopyranoside, kaempferol 3-O-β-rutinoside, kaempferol 3-O-D-galactoside, kaempferol 7-O-neohesperidoside, kaempferol 3-rutinoside 4′-glucoside, kaempferitrin, robinin, afzelin, and leucoside), isorhamnetin (as well as its derivates and metabolites such as isorhammetin-3-O-β-D-glucotrioside and isorhammetin 3-O-glucoside), naringenin (as well as its metabolites and derivates such as naringenin 4′,7-dimethyl ether, naringenin-6-C-glucoside, narirutin, 5-O-methylnaringenin, and prunin), myricetin (as well as its derivates and metabolites such as myricitrin, myricetin 3-O-rutinoside, and myricetin 3-rutinoside-7-glucoside), baicalein (as well as its derivates and metabolites such as baicalin, baicalin hydrate, and biochanin A), glycitein, 5,6,7-dihydroxyflavone, fisetin, caffeic acid phenethyl ester, octyl gallate, lauryl gallate, lignan, enterolactone, kolaflavanone, kolaviron, and secoisolariciresinol diglucoside. In some embodiments, the composition comprises about 0.05-6.0% by volume of the 5-alpha reductase modulator. The total amount of 5-alpha reductase modulator in the composition, by volume, may be about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, or any range including and/or in between any two of these values. If the composition is intended for a male, the composition may preferably include about 0.1% to about 0.6% by volume of the 5-alpha reductase modulator (or any range as recited above). If the composition is intended for a female, the composition may preferably include about 1.0% to about 6.0% by volume of the 5-alpha reductase modulator (or any range as recited above).

In some embodiments, the 5-alpha reductase modulator comprises one or more of from emodin, emodin 8-glucoside, and aloe-emodin. In some embodiments, the composition comprises about 0.2-3.0% or about 0.25-2.5% by volume of emodin. In some embodiments, the composition comprises about 0.25%, about 0.3%, about 0.4%, about 0.5%, about 0.6%, about 0.8%, about 1.0%, about 1.25%, about 1.5%, about 1.75%, about 2.0%, about 2.25%, about 2.5%, about 2.75%, or about 3.0% (or any range including and/or in between any two of these values) by volume of emodin. In some embodiments, the composition comprises about 0.5% by volume of emodin.

In some embodiments, the 5-alpha reductase modulator comprises finasteride. In some embodiments, the composition comprises about 0.25-0.75% by volume of finasteride. In some embodiments, the composition comprises about 0.25% or about 0.5% by volume of finasteride. In some embodiments, the composition comprises about 0.5% by volume of finasteride.

In some embodiments, the 5-alpha reductase modulator comprises eugenol. In some embodiments, the composition comprises about 0.1-1.75%, about 0.5-1.75%, about 0.5-2.5%, or about 0.5-2.0%, by volume of eugenol. In some embodiments, the composition comprises about 0.1-1.75% by volume of eugenol. In some embodiments, the composition comprises about 0.05-0.2% by volume of eugenol. In some embodiments, the composition comprises about 0.07-0.15% by volume of eugenol. In some embodiments, the composition comprises about 0.05%, about 0.06%, about 0.07%, about 0.08%, about 0.09%, about 0.1%, about 0.25%, about 0.5%, about 0.75%, about 1.0%, about 1.25%, about 1.5%, about 1.75%, or about 2.0% (or any range including and/or in between any two of these values) by volume of eugenol. In some embodiments, the composition comprises about 0.1% by volume of eugenol.

In some embodiments, the 5-alpha reductase modulator comprises dutasteride. In some embodiments, the composition comprises about 0.05-0.2% by volume of dutasteride. In some embodiments, the composition comprises about 0.07-0.15% by volume of dutasteride. In some embodiments, the composition comprises about 0.05%, about 0.06%, about 0.07%, about 0.08%, about 0.09%, or about 0.1% (or any range including and/or in between any two of these values) by volume of dutasteride. In some embodiments, the composition comprises about 0.1% by volume of dutasteride.

In some embodiments, the 5-alpha reductase modulator comprises two, three, four, five, six, or all of the group selected from emodin (as well as its metabolites and derivates such as emodin 8-glucoside and aloe-emodin), finasteride, eugenol, dutasteride, enoxolone, equol (such as (S)-equol,(R)-equol, as well as a racemic mixture of equol), 3-(4-hydroxyphenyl)chroman-4,7-diol, genistein (as well as its derivates and metabolites such as genisitin), sophoricoside, daizein (as well as its metabolites such as daidzin), 1,2-dimyristoyl-sn-glycero-3-phospo-L-serine sodium salt, kaempferol (as well as its methobolites or derivates such as kaempferol, kaempferol 3-glucoside, kaempferol 7-O-β-D-glucopyranoside, kaempferol 3-O-β-rutinoside, kaempferol 3-O-D-galactoside, kaempferol 7-O-neohesperidoside, kaempferol 3-rutinoside 4′-glucoside, kaempferitrin, robinin, afzelin, and leucoside), isorhamnetin (as well as its derivates and metabolites such as isorhammetin-3-O-β-D-glucotrioside and isorhammetin 3-O-glucoside), naringenin (as well as its metabolites and derivates such as naringenin 4′,7-dimethyl ether, naringenin-6-C-glucoside, narirutin, 5-O-methylnaringenin, and prunin), myricetin (as well as its derivates and metabolites such as myricitrin, myricetin 3-O-rutinoside, and myricetin 3-rutinoside-7-glucoside), baicalein (as well as its derivates and metabolites such as baicalin, baicalin hydrate, and biochanin A), glycitein, 5,6,7-dihydroxyflavone, fisetin, caffeic acid phenethyl ester, octyl gallate, lauryl gallate, lignan, enterolactone, kolaflavanone, kolaviron, and secoisolariciresinol diglucoside.

In some embodiments, the composition provided herein further comprises non-terminal antioxidants or non-suicidal antioxidants. Non-limiting illustrative examples of a non-terminal antioxidant or non-suicidal antioxidant include vitamin A, vitamin C, vitamin E, beta-carotene, alpha-lipoic acid, and ferulic acid. In some embodiments, the non-terminal antioxidant or non-suicidal antioxidant comprises one or more of a group of vitamin A, vitamin C, vitamin E, beta-carotene, alpha-lipoic acid, and ferulic acid. In some embodiments, the composition comprises about 0.01-0.8%, 0.05-0.8%, about 0.05-0.5%, about 0.01-0.05%, about 0.05-0.2%, about 0.1-0.8%, about 0.1-0.3%, or about 0.25-0.5% by volume of the non-terminal antioxidant or non-suicidal antioxidant. In some embodiments, the composition comprises about 0.05%, 0.1%, 0.2%, 0.25%, 0.3%, 0.5%, or 0.75% (or any range including and/or in between any two of these values) by volume of the non-terminal antioxidant or non-suicidal antioxidant.

In some embodiments, the non-terminal or non-suicidal antioxidant comprises vitamin A. In some embodiments, the composition comprises about 0.01-0.3% by volume of vitamin A. In some embodiments, the composition comprises about 0.01%, about 0.05%, about 0.1%, about 0.2%, about 0.25%, or about 0.3% by volume of vitamin A, or any range including and/or in-between any two of these values. In some embodiments, the composition comprises about 0.25% by volume of vitamin A.

In some embodiments, the non-terminal or non-suicidal antioxidant comprises vitamin C. In some embodiments, the composition comprises about 0.1-0.5% by volume of vitamin C. In some embodiments, the composition comprises about 0.1%, about 0.2%, about 0.25%, about 0.3%, about 0.4%, or about 0.5% (or any range including and/or in-between any two of these values) by volume of vitamin C. In some embodiments, the composition comprises about 0.1% of vitamin C. In some embodiments, the composition comprises about 0.25% by volume of vitamin C.

In some embodiments, the non-terminal or non-suicidal antioxidant comprises vitamin E. In some embodiments, the composition comprises about 0.1-0.5% by volume of vitamin E. In some embodiments, the composition comprises about 0.1%, about 0.2%, about 0.25%, about 0.3%, about 0.4%, or about 0.5% (or any range including and/or in-between any two of these values) by volume of vitamin E. In some embodiments, the composition comprises about 0.1% of vitamin E. In some embodiments, the composition comprises about 0.25% by volume of vitamin E.

In some embodiments, the non-terminal or non-suicidal antioxidant comprises beta-carotene. In some embodiments, the composition comprises about 0.01-0.05%, about 0.05-0.5%, about 0.05-0.2%, or about 0.01-0.3% by volume of beta-carotene. In some embodiments, the composition comprises about 0.01%, about 0.05%, about 0.06%, about 0.07%, about 0.08%, about 0.1%, about 0.2%, or about 0.3% (or any range including and/or in-between any two of these values) by volume of beta-carotene. In some embodiments, the composition comprises about 0.05% by volume of beta-carotene.

In some embodiments, the non-terminal or non-suicidal antioxidant comprises alpha-lipoic acid. In some embodiments, the composition comprises about 0.1-0.3% by volume of alpha-lipoic acid. In some embodiments, the composition comprises about 0.1%, about 0.2%, about 0.25%, or about 0.3% (or any range including and/or in between any two of these values) by volume of alpha-lipoic acid. In some embodiments, the composition comprises about 0.25% by volume of alpha-lipoic acid.

In some embodiments, the non-terminal antioxidant or non-suicidal antioxidant comprises ferulic acid. In some embodiments, the composition comprises about 0.01-0.3% by volume of ferulic acid. In some embodiments, the composition comprises about 0.01%, about 0.05%, about 0.1%, about 0.2%, about 0.25%, or about 0.3% (or any range including and/or in-between any two of these values) by volume of ferulic acid. In some embodiments, the composition comprises about 0.25% by volume of ferulic acid.

In some embodiments, the non-terminal antioxidant or non-suicidal antioxidant comprises two, three, four, five, or all of a group of vitamin A, vitamin C, vitamin E, beta-carotene, alpha-lipoic acid, and ferulic acid.

In some embodiments, the non-terminal antioxidant or non-suicidal antioxidant comprises vitamin C and vitamin E. In some embodiments, the composition comprises about 0.1-0.8%, about 0.1-0.3%, or about 0.25-0.5% by volume of vitamin C and vitamin E. In some embodiments, the composition comprises about 0.25%, 0.5%, or about 0.75% (or any range including and/or in between any two of these values) by volume of vitamin C and vitamin E. In some embodiments, the composition comprises about 0.5% by volume of vitamin C and vitamin E.

In some embodiments, the composition provided herein further comprises one or more of a group selected from the terminal antioxidant or suicidal antioxidant as described above, the moisturizer as described above, the anti-fungal agent as described above, the UV filter as described above, the piloerector muscle relaxer as described above, the blood circulator as described above, the 5-alpha reductase modulator as described above, and the non-terminal antioxidant or non-suicidal antioxidant as described above. In some embodiments, the composition provided herein further comprises two, three, four, five, six, seven, or all of a group selected from the terminal or suicidal antioxidant as described above, the moisturizer as described above, the anti-fungal agent as described above, the UV filter as described above, the piloerector muscle relaxer as described above, the blood circulator as described above, the 5-alpha reductase modulator as described above, and the non-terminal antioxidant or non-suicidal antioxidant as described above.

In some embodiments, the composition provided herein further comprises the terminal antioxidant or suicidal antioxidant as described above, the moisturizer as described above, the antifungal agent as described above, the UV filter as described above, the piloerector muscle relaxer as described above, the blood circulator as described above, the 5-alpha reductase modulator as described above, and the non-terminal antioxidant or non-suicidal antioxidant as described above.

In some embodiments, the composition provided herein further comprises the terminal antioxidant or suicidal antioxidant as described above, the moisturizer as described above, the antifungal agent as described above, the UV filter as described above, the blood circulator as described above, the 5-alpha reductase modulator as described above, and the non-terminal antioxidant or non-suicidal antioxidant as described above.

In some embodiments, the composition provided herein further comprises the moisturizer as described above, the antifungal agent as described above, the UV filter as described above, the piloerector muscle relaxer as described above, the blood circulator as described above, the 5-alpha reductase modulator as described above, and the non-terminal antioxidant or non-suicidal antioxidant as described above.

In some embodiments, the composition provided herein further comprises the moisturizer as described above, the antifungal agent as described above, the UV filter as described above, the blood circulator as described above, the 5-alpha reductase modulator as described above, and the non-terminal antioxidant or non-suicidal antioxidant as described above.

In some embodiments, the composition provided herein further comprises the moisturizer as described above, the antifungal agent as described above, the UV filter as described above, the blood circulator as described above, and the non-terminal antioxidant or non-suicidal antioxidant as described above.

In some embodiments, the composition provided herein further comprises the moisturizer as described above, the UV filter as described above, the blood circulator as described above, and the non-terminal antioxidant or non-suicidal antioxidant as described above.

In some embodiments, the composition provided herein further comprises one or more of a group of copper peptide, vitamin B1, vitamin B2, vitamin B3, vitamin B5, vitamin B6, vitamin B7, vitamin B9, vitamin B12, bimatoprost, tafluprost, travopost, latanoprost, caffeine, clove oil, and St. John's wort oil.

In some embodiments, the composition further comprises copper peptide. In some embodiments, the composition comprises about 0.05-0.2% by volume of copper peptide. In some embodiments, the composition comprises about 0.07-0.15% by volume of copper peptide. In some embodiments, the composition comprises about 0.05%, about 0.06%, about 0.07%, about 0.08%, about 0.09%, or about 0.1% (or any range including and/or in between any two of these values) by volume of copper peptide. In some embodiments, the composition comprises about 0.1% by volume of copper peptide.

In some embodiments, the composition further comprises vitamin B1. In some embodiments, the composition comprises about 0.1-3%, about 0.3-3%, about 0.5-3%, about 1-3%, about 1.5-3%, or about 2-3% by volume of vitamin B1. In some embodiments, the composition comprises about 0.1-3% by volume of vitamin B1.

In some embodiments, the composition further comprises vitamin B2. In some embodiments, the composition comprises about 0.1-3%, about 0.3-3%, about 0.5-3%, about 1-3%, about 1.5-3%, or about 2-3% (or any range including and/or in between any two of these values) by volume of vitamin B2. In some embodiments, the composition comprises about 0.1-3% by volume of vitamin B2.

In some embodiments, the composition further comprises vitamin B3. In some embodiments, the composition comprises about 0.1-3%, about 0.3-3%, about 0.5-3%, about 1-3%, about 1.5-3%, or about 2-3% (or any range including and/or in between any two of these values) by volume of vitamin B3. In some embodiments, the composition comprises about 0.1-3% by volume of vitamin B3.

In some embodiments, the composition further comprises vitamin B5. In some embodiments, the composition comprises about 0.1-3%, about 0.3-3%, about 0.5-3%, about 1-3%, about 1.5-3%, or about 2-3% (or any range including and/or in between any two of these values) by volume of vitamin B5. In some embodiments, the composition comprises about 0.1-3% by volume of vitamin B5.

In some embodiments, the composition further comprises vitamin B6. In some embodiments, the composition comprises about 0.1-3%, about 0.3-3%, about 0.5-3%, about 1-3%, about 1.5-3%, or about 2-3% (or any range including and/or in between any two of these values) by volume of vitamin B6. In some embodiments, the composition comprises about 0.1-3% by volume of vitamin B6.

In some embodiments, the composition further comprises vitamin B7. In some embodiments, the composition comprises about 0.1-3%, about 0.3-3%, about 0.5-3%, about 1-3%, about 1.5-3%, or about 2-3% (or any range including and/or in between any two of these values) by volume of vitamin B7. In some embodiments, the composition comprises about 0.1-3% by volume of vitamin B7.

In some embodiments, the composition further comprises vitamin B9. In some embodiments, the composition comprises about 0.1-3%, about 0.3-3%, about 0.5-3%, about 1-3%, about 1.5-3%, or about 2-3% (or any range including and/or in between any two of these values) by volume of vitamin B9. In some embodiments, the composition comprises about 0.1-3% by volume of vitamin B9.

In some embodiments, the composition further comprises vitamin B12. In some embodiments, the composition comprises about 0.1-3%, about 0.3-3%, about 0.5-3%, about 1-3%, about 1.5-3%, or about 2-3% (or any range including and/or in between any two of these values) by volume of vitamin B12. In some embodiments, the composition comprises about 0.1-3% by volume of vitamin B12.

In some embodiments, the composition further comprises bimatoprost. In some embodiments, the composition comprises about 0.01-1%, about 0.1-1%, about 0.3-1%, or about 0.6-1% (or any range including and/or in between any two of these values) by volume of bimatoprost. In some embodiments, the composition comprises about 0.01-1% by volume of bimatoprost.

In some embodiments, the composition further comprises tafluprost. In some embodiments, the composition comprises about 0.01-1%, about 0.1-1%, about 0.3-1%, or about 0.6-1% (or any range including and/or in between any two of these values) by volume of tafluprost. In some embodiments, the composition comprises about 0.01-1% by volume of tafluprost.

In some embodiments, the composition further comprises travopost. In some embodiments, the composition comprises about 0.01-1%, about 0.1-1%, about 0.3-1%, or about 0.6-1% (or any range including and/or in between any two of these values) by volume of travopost. In some embodiments, the composition comprises about 0.01-1% by volume of travopost.

In some embodiments, the composition further comprises latanoprost. In some embodiments, the composition comprises about 0.01-1%, about 0.1-1%, about 0.3-1%, or about 0.6-1% (or any range including and/or in between any two of these values) by volume of latanoprost. In some embodiments, the composition comprises about 0.01-1% by volume of latanoprost.

In some embodiments, the composition further comprises caffeine. In some embodiments, the composition comprises about 0.1-3%, about 0.3-3%, about 0.5-3%, about 1-3%, about 1.5-3%, or about 2-3% (or any range including and/or in between any two of these values) by volume of caffeine. In some embodiments, the composition comprises about 0.1-3% by volume of caffeine.

In some embodiments, the composition further comprises clove oil. In some embodiments, the composition comprises about 0.01-3% by volume of clove oil. The composition may comprise about 0.01%, about 0.03%, about 0.05%, about 0.1%, about 0.15%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.4%, about 2.8%, or about 3% (or any range including and/or in-between any two of these values) by volume of clove oil.

In some embodiments, the composition further comprises St. John's wort oil. In some embodiments, the composition comprises about 0.01-3% by volume of St. John's wort oil The composition may comprise about 0.01%, about 0.03%, about 0.05%, about 0.1%, about 0.15%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.4%, about 2.8%, or about 3% (or any range including and/or in-between any two of these values) by volume of St. John's wort oil.

In some embodiments, the composition provided herein further comprises two, three, four, five, six, seven, eight, nine, ten, eleven, twelve, thirteen, fourteen, fifteen, or all of a group of copper peptide, vitamin B1, vitamin B2, vitamin B3, vitamin B5, vitamin B6, vitamin B7, vitamin B9, vitamin B12, bimatoprost, tafluprost, travopost, latanoprost, caffeine, clove oil, and St. John's wort oil.

In some embodiments, the composition further comprises one or more of a group of vitamin B1, vitamin B2, vitamin B3, vitamin B5, vitamin B6, vitamin B7, vitamin B9, and vitamin B12. In some embodiments, the composition comprises about 0.1-3%, about 0.3-3%, about 0.5-3%, about 1-3%, about 1.5-3%, or about 2-3% (or any range including and/or in between any two of these values) by volume of the one or more of a group of vitamin B1, vitamin B2, vitamin B3, vitamin B5, vitamin B6, vitamin B7, vitamin B9, and vitamin B12. In some embodiments, the composition comprises about 0.1-3% by volume of the one or more of a group of vitamin B1, vitamin B2, vitamin B3, vitamin B5, vitamin B6, vitamin B7, vitamin B9, and vitamin B12.

In some embodiments, the composition further comprises one or more of a group of copper peptide, clove oil, and St. John's oil. In some embodiments, the composition further comprises copper peptide and clove oil. In some embodiments, the composition further comprises copper peptide and St. John's wort oil. In some embodiments, the composition further comprises clove oil and St. John's wort oil. In some embodiments, the composition further comprises copper peptide, clove oil, and St. John's wort oil.

In some embodiments, the composition provided herein further comprises the terminal antioxidant or suicidal antioxidant as described above, the moisturizer as described above, the antifungal agent as described above, the UV filter as described above, the piloerector muscle relaxer as described above, the blood circulator as described above, the 5-alpha reductase modulator as described above, the non-terminal antioxidant or non-suicidal antioxidant as described above, copper peptide, and clove oil.

In some embodiments, the composition provided herein further comprises the terminal antioxidant or suicidal antioxidant as described above, the moisturizer as described above, the antifungal agent as described above, the UV filter as described above, the blood circulator as described above, the 5-alpha reductase modulator as described above, the non-terminal antioxidant or non-suicidal antioxidant as described above, copper peptide, and clove oil.

In some embodiments, the composition provided herein further comprises the moisturizer as described above, the antifungal agent as described above, the UV filter as described above, the piloerector muscle relaxer as described above, the blood circulator as described above, the 5-alpha reductase modulator as described above, the non-terminal antioxidant or non-suicidal antioxidant as described above, copper peptide, and clove oil.

In some embodiments, the composition provided herein further comprises the moisturizer as described above, the antifungal agent as described above, the UV filter as described above, the blood circulator as described above, the 5-alpha reductase modulator as described above, and the non-terminal antioxidant or non-suicidal antioxidant as described above, copper peptide, and clove oil.

In some embodiments, the composition provided herein further comprises the moisturizer as described above, the antifungal agent as described above, the UV filter as described above, the blood circulator as described above, the non-terminal antioxidant or non-suicidal antioxidant as described above, and clove oil.

In some embodiments, the composition provided herein further comprises the moisturizer as described above, the UV filter as described above, the blood circulator as described above, the non-terminal antioxidant or non-suicidal antioxidant as described above, and clove oil.

In some embodiments, the composition provided herein further comprises the terminal antioxidant or suicidal antioxidant as described above, the moisturizer as described above, the antifungal agent as described above, the UV filter as described above, the piloerector muscle relaxer as described above, the blood circulator as described above, the 5-alpha reductase modulator as described above, the non-terminal antioxidant or non-suicidal antioxidant as described above, copper peptide, clove oil, and St. John's wort oil.

In some embodiments, the composition provided herein further comprises the terminal antioxidant or suicidal antioxidant as described above, the moisturizer as described above, the antifungal agent as described above, the UV filter as described above, the blood circulator as described above, the 5-alpha reductase modulator as described above, the non-terminal antioxidant or non-suicidal antioxidant as described above, copper peptide, clove oil, and St. John's wort oil.

In some embodiments, the composition provided herein further comprises the moisturizer as described above, the antifungal agent as described above, the UV filter as described above, the piloerector muscle relaxer as described above, the blood circulator as described above, the 5-alpha reductase modulator as described above, the non-terminal antioxidant or non-suicidal antioxidant as described above, copper peptide, clove oil, and St. John's wort oil.

In some embodiments, the composition provided herein further comprises the moisturizer as described above, the antifungal agent as described above, the UV filter as described above, the blood circulator as described above, the 5-alpha reductase modulator as described above, and the non-terminal antioxidant or non-suicidal antioxidant as described above, copper peptide, clove oil, and St. John's wort oil.

In some embodiments, the composition provided herein further comprises the moisturizer as described above, the antifungal agent as described above, the UV filter as described above, the blood circulator as described above, the non-terminal antioxidant or non-suicidal antioxidant as described above, clove oil, and St. John's wort oil.

In some embodiments, the composition provided herein further comprises the moisturizer as described above, the UV filter as described above, the blood circulator as described above, the non-terminal antioxidant or non-suicidal antioxidant as described above, clove oil, and St. John's wort oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen or all of a group of urea, allicin, eugenol, zinc oxide, beta-carotene, minoxidil, pyrrolidinyl diaminopyrimidine oxide, arginine, capsaicin, finasteride, emodin, vitamin C, vitamin E, copper peptide, and clove oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, finasteride, emodin, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, minoxidil, arginine, capsaicin, finasteride, vitamin C, vitamin E, and copper peptide. In some embodiments, the epigenetic modifier comprises melatonin and EGCG and the composition further comprises at least two, three, four, five, six, seven, eight, nine, ten, eleven, twelve, or all of a group of urea, allicin, eugenol, minoxidil, zinc oxide, beta-carotene, arginine, capsaicin, finasteride, vitamin C, vitamin E, copper peptide, and clove oil.

In some embodiments, the epigenetic modifier comprises melatonin and EGCG and the composition further comprises urea, allicin, eugenol, zinc oxide, beta-carotene, minoxidil, arginine, capsaicin, finasteride, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, pyrrolidinyl diaminopyrimidine oxide, arginine, capsaicin, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, or all of a group of urea, allicin, eugenol, zinc oxide, beta-carotene, pyrrolidinyl diaminopyrimidine oxide, arginine, capsaicin, emodin, vitamin C, vitamin E, copper peptide, and clove oil.

In some embodiments, the epigenetic modifier comprises melatonin and EGCG and the composition further comprises urea, allicin, eugenol, zinc oxide, beta-carotene, pyrrolidinyl diaminopyrimidine oxide, arginine, capsaicin, emodin, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, finasteride, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of the group of urea, allicin, eugenol, zinc oxide, beta-carotene, minoxidil, pyrrolidinyl diaminopyrimidine oxide, arginine, capsaicin, finasteride, vitamin C, vitamin E, copper peptide, and clove oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, finasteride, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of the group of urea, allicin, eugenol, zinc oxide, beta-carotene, minoxidil, pyrrolidinyl diaminopyrimidine oxide, arginine, capsaicin, emodin, vitamin C, vitamin E, copper peptide, and clove oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, emodin, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, minoxidil, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of a group of urea, allicin, eugenol, zinc oxide, beta-carotene, minoxidil, arginine, capsaicin, finasteride, emodin, vitamin C, vitamin E, copper peptide, and clove oil.

In some embodiments, the epigenetic modifier comprises melatonin and EGCG and the composition further comprises urea, allicin, eugenol, zinc oxide, beta-carotene, minoxidil, arginine, capsaicin, finasteride, emodin, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, pyrrolidinyl diaminopyrimidine oxide, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of a group of urea, allicin, eugenol, zinc oxide, beta-carotene, pyrrolidinyl diaminopyrimidine oxide, arginine, capsaicin, finasteride, emodin, vitamin C, vitamin E, copper peptide, and clove oil.

In some embodiments, the epigenetic modifier comprises melatonin and EGCG and the composition further comprises urea, allicin, eugenol, zinc oxide, beta-carotene, pyrrolidinyl diaminopyrimidine oxide, arginine, capsaicin, finasteride, emodin, vitamin C, vitamin E, copper peptide, and clove oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen, fifteen, or all of a group of urea, allicin, eugenol, zinc oxide, beta-carotene, minoxidil, pyrrolidinyl diaminopyrimidine oxide, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, minoxidil, arginine, capsaicin, finasteride, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of the group of urea, allicin, eugenol, zinc oxide, beta-carotene, minoxidil, arginine, capsaicin, finasteride, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, minoxidil, arginine, capsaicin, finasteride, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, pyrrolidinyl diaminopyrimidine oxide, arginine, capsaicin, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, or all of a group of urea, allicin, eugenol, zinc oxide, beta-carotene, pyrrolidinyl diaminopyrimidine oxide, arginine, capsaicin, emodin, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, pyrrolidinyl diaminopyrimidine oxide, arginine, capsaicin, emodin, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, finasteride, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen, or all of a group of urea, allicin, eugenol, zinc oxide, beta-carotene, minoxidil, pyrrolidinyl diaminopyrimidine oxide, arginine, capsaicin, finasteride, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, finasteride, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, minoxidil, pyrrolidinyl diaminopyrimidine oxide, arginine, capsaicin, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen, or all of the group of urea, allicin, eugenol, zinc oxide, beta-carotene, minoxidil, pyrrolidinyl diaminopyrimidine oxide, arginine, capsaicin, emodin, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, minoxidil, pyrrolidinyl diaminopyrimidine oxide, arginine, capsaicin, emodin, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, minoxidil, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen, or all of a group of urea, allicin, eugenol, zinc oxide, beta-carotene, minoxidil, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, minoxidil, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises at least one or more of a group of urea, allicin, eugenol, zinc oxide, beta-carotene, pyrrolidinyl diaminopyrimidine oxide, arginine, capsaicin, finasteride, emodin, vitamin C, vitamin E, and copper peptide. In some embodiments, the epigenetic modifier comprises melatonin and EGCG and the composition further comprises at least two, three, four, five, six, seven, eight, nine, ten, eleven, twelve, thirteen, fourteen, or all of a group of urea, allicin, eugenol, zinc oxide, beta-carotene, pyrrolidinyl diaminopyrimidine oxide, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises urea, allicin, eugenol, zinc oxide, beta-carotene, pyrrolidinyl diaminopyrimidine oxide, arginine, capsaicin, finasteride, emodin, vitamin C, vitamin E, copper peptide, clove oil, and St. John's wort oil.

In some embodiments, the epigenetic modifier comprises melatonin and EGCG and the composition further comprises urea, zinc oxide, beta-carotene, minoxidil, pyrrolidinyl diaminopyrimidine oxide, arginine, vitamin E, clove oil, and St. John's oil.

In some embodiments, the epigenetic modifier comprises melatonin and EGCG and the composition further comprises urea, zinc oxide, beta-carotene, minoxidil, arginine, vitamin E, clove oil, and St. John's oil.

In some embodiments, the epigenetic modifier comprises melatonin and EGCG and the composition further comprises urea, zinc oxide, beta-carotene, pyrrolidinyl diaminopyrimidine oxide, arginine, vitamin E, clove oil, and St. John's oil.

In some embodiments, the epigenetic modifier comprises melatonin and EGCG and the composition further comprises urea, allicin, zinc oxide, beta-carotene, minoxidil, pyrrolidinyl diaminopyrimidine oxide, arginine, capsaicin, vitamin C, vitamin E, clove oil, and St. John's oil.

In some embodiments, the epigenetic modifier comprises melatonin and EGCG and the composition further comprises urea, allicin, zinc oxide, beta-carotene, minoxidil, arginine, capsaicin, vitamin C, vitamin E, clove oil, and St. John's oil.

In some embodiments, the epigenetic modifier comprises melatonin and EGCG and the composition further comprises urea, allicin, zinc oxide, beta-carotene, pyrrolidinyl diaminopyrimidine oxide, arginine, capsaicin, vitamin C, vitamin E, clove oil, and St. John's oil.

As noted above, the composition provided herein comprises a cosmetically acceptable carrier. In some embodiments, the cosmetically acceptable carrier comprises an organic solvent, a polyol, water, and an oil.

Organic solvents useful in cosmetically acceptable carriers are well-known in the art. Non-limiting illustrative examples of organic solvents that may be used in cosmetically acceptable carriers include dipropyleneglycol and monopropyleneglycol. The composition may include from about 1% to about 50% by volume organic solvent. Thus, the composition in any embodiment herein may include about 1%, about 3%, about 5%, about 10%, about 15%, about 20%, about 25%, about 30%, about 35%, about 40%, about 45%, about 50% (or any range including and/or in between any two of these values) by volume organic solvent. In some embodiments, the composition comprises about 15-45% by volume of the organic solvent. In some embodiments, the composition comprises about 20-40% or about 25-35% by volume of the organic solvent.

In some embodiments, the organic solvent comprises dipropyleneglycol. In some embodiments, the organic solvent comprises monopropyleneglycol. In some embodiments, the organic solvent comprises dipropyleneglycol and monopropyleneglycol. In some embodiments, the dipropyleneglycol, the monopropyleneglycol, or the combination of dipropyleneglycol and monopropyleneglycol comprises about 15-45% or about 20-40% by volume of the composition. In some embodiments, the dipropyleneglycol comprises about 20-40% by volume of the composition. In some embodiments, the dipropyleneglycol comprises about 15%, about 20%, about 25%, about 30%, about 35%, about 40%, or about 45% (or any range including and/or in between any two of these values) by volume of the composition. In some embodiments, the monopropyleneglycol comprises about 15%, about 20%, about 25%, about 30%, about 35%, about 40%, or about 45% (or any range including and/or in between any two of these values) by volume of the composition.

The cosmetically acceptable carrier of the compositions disclosed herein may also include polyols. Polyols useful in cosmetically acceptable carriers are well-known in the art. Non-limiting illustrative examples of polyols include glycerin, sorbitol, and xylitol. In some embodiments, the composition comprises about 1-50% or about 1-20% by volume of the polyol. In some embodiments, the composition comprises about 1%, about 3%, about 5%, about 10%, about 15%, about 20%, or about 25% by volume of the polyol.

In some embodiments, the polyol comprises glycerin. In some embodiments, the composition comprises about 1-50% by volume of glycerin. In some embodiments, the composition comprises about 1-25% by volume of glycerin. In some embodiments, the composition comprises about 1-20% by volume of glycerin. In some embodiments, the composition comprises about 1%, about 3%, about 5%, about 10%, about 15%, about 20%, about 25%, about 30%, about 35%, about 40%, about 45%, about 50% (or any range including and/or in between any two of these values) by volume of glycerin.

The cosmetically acceptable carrier of the compositions disclosed herein also include water. In some embodiments, the composition comprises about 15-45% or about 20-40% by volume of water. In some embodiments, the composition comprises about 15%, about 20%, about 25%, about 30%, about 35%, about 40%, or about 45% by volume of water. In some embodiments, the water comprises distilled water. In some embodiments, the water comprises deionized water.

The cosmetically acceptable carrier of the compositions disclosed herein also include an oil. In some embodiments, the composition comprises about 1% to about 35% by volume of the oil. Thus, the composition of any embodiment herein may include about 1%, about 2%, about 3%, or about 4%, about 5%, about 6%, about 7%, about 8%, about 9%, about 10%, about 15%, about 20%, about 25%, about 30%, about 35%, about 40%, about 45%, about 50% (or any range including and/or in between any two of these values) by volume of the oil. For example, in some embodiments the composition includes about 1% to about 10% by volume of the oil.

In some embodiments, the oil comprises one or more of a group of olive oil, rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil. In some embodiments, the oil comprises two, three, four, five, six, seven, eight, nine, ten, eleven, twelve, thirteen, fourteen, fifteen, sixteen, seventeen, or all of the group of olive oil, rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil.

In some embodiments, the oil comprises one or more of a group of olive oil, clove oil, and St. John's wort oil. In some embodiments, the oil comprises olive oil and St. John's wort oil. In some embodiments, the oil comprises olive oil and clove oil. In some embodiments, the oil comprises clove oil and St. John's wort oil. In some embodiments, the oil comprises olive oil, clove oil, and St. John's wort oil.

In some embodiments, the oil comprises olive oil. In some embodiments, the oil comprises rosemary oil. In some embodiments, the oil comprises sweet almond oil. In some embodiments, the oil comprises nigella sativa oil. In some embodiments, the oil comprises St. John's wort oil. In some embodiments, the oil comprises argan oil. In some embodiments, the oil comprises theme oil. In some embodiments, the oil comprises pumpkin seed oil. In some embodiments, the oil comprises black marble oil. In some embodiments, the oil comprises clove oil. In some embodiments, the oil comprises red palm oil. In some embodiments, the oil comprises horse tail oil. In some embodiments, the oil comprises coconut oil. In some embodiments, the oil comprises walnut oil. In some embodiments, the oil comprises pine oil. In some embodiments, the oil comprises nettle seed oil. In some embodiments, the oil comprises castor oil. In some embodiments, the oil comprises hazelnut oil.

In some embodiments, the oil comprises about 45-99% by volume of olive oil. In some embodiments, the oil comprises about 49-98% by volume of olive oil. In some embodiments, the oil comprises about 50-98% or about 50-99% by volume of olive oil. In some embodiments, the oil comprises about 45%, about 50%, about 55%, about 60%, about 65%, about 70%, about 75%, about 80%, about 85%, about 90%, about 95%, about 96%, about 97%, about 98%, or about 99% (or any range including and/or in between any two of these values) by volume of olive oil.

In some embodiments, the oil comprises about 0.01-55%, about 0.05%-55%, about 0.1%-55%, about 0.5-55%, or about 1-55% (or any range including and/or in between any two of these values) by volume of one or more of a group of rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil.

In some embodiments, the oil comprises about 0.01-0.1%, about 0.05-0.1%, about 0.01-1%, about 0.05-1%, about 0.01-2%, about 0.05-2%, 0.01-3%, about 0.05-3%, about 0.1-1%, about 0.1-2%, or about 0.1-3% (or any range including and/or in between any two of these values) by volume of any of a group of rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil. In some embodiments, the oil comprises about 0.01-0.1%, about 0.05-0.1%, or about 0.1-3% by volume of any of a group of rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil.

In some embodiments, the oil comprises about 0.01-0.1%, or about 0.05-0.1% of clove oil. In some embodiments, the oil comprises about 0.01% by volume of clove oil.

In some embodiments, the oil comprises about 0.01-0.1%, or about 0.05-0.1% of St. John's wort oil. In some embodiments, oil comprises about 0.05%, 0.07%, 0.09%, or 0.1% of St. John's wort oil.

In some embodiments, the composition provided herein further comprises one or more of a cosmetically acceptable excipient selected from a group of an emulsifier, an anti-microbial agent, a pH regulator, a preservative, and a fragrance. Other cosmetic excipients are well known in the art, for example, those described in “Remington: The Science & Practice of Pharmacy”, 19th ed., Williams & Williams, (1995), the “Physician's Desk Reference”, 52.sup.nd ed., Medical Economics, Montvale, N.J. (1998), and Kibbe, A. H., Handbook of Pharmaceutical Excipients, 3.sup.rd Edition, American Pharmaceutical Association, Washington, D.C., 2000.

In some embodiments, the composition further comprises an emulsifier. An emulsifier is used to homogenize an emulsion between an oil phase and an aqueous phase. Non-limiting illustrative examples of an emulsifier include polysorbates 20, polysorbates 50, polysorbates 80, polyethylene glycol (PEG) 40, PEG 100, cetyl alcohol, ceteareth 20, ceteareth 25, and lecithin. In some embodiments, the emulsifier is added in amounts to obtain the desired homogeny between an oil phase and an aqueous phase. In any embodiment including an emulsifier, the composition may include about 5%, about 10%, about 15%, or about 20% (or any range including and/or in-between any two of these values) by volume of emulsifier.

In some embodiments, the composition provided herein further comprises an anti-microbial agent. Non-limiting illustrative examples of an anti-microbial agent include sorbate salts (such as sodium sorbate, potassium sorbate, and calcium sorbate), a benzoate salt (such as sodium benzoate and potassium benzoate), and nisin.

In some embodiments, the composition further comprises a pH regulator. Non-limiting illustrative examples of a pH regulator include citric acid, vitamin C, acetic acid, sodium hydroxide, and potassium hydroxide.

In some embodiments, the composition further comprises a preservative. Non-limiting illustrative examples of a preservative include potassium sorbate, methyl-p-hydroxybenzoate, C₁₂ to C₁₅ alkyl benzoates, alkyl p-hydroxybenzoates, propyl and butyl p-hydroxybenzoates, aloe vera extract, ascorbic acid, benzalkonium chloride, benzoic acid, benzoic acid esters of C₉ to C₁₅ alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, silicone oils, sodium propionate, sodium benzoate, sodium bisulfite, disodium metabisulfite, sorbic acid, stearic fatty acid, vitamin E, vitamin E acetate and derivatives, esters, salts and mixtures thereof.

In some embodiments, the composition further comprises a fragrance. Non-limiting illustrative examples of a fragrance include but are not limited to limonene, menthol, and essences of vanilla, coffee, apple, and strawberry.

In some embodiments, the composition provided herein includes Compositions #1-64 as described in Tables 1-6, wherein Compositions #1-64 further comprise the cosmetically acceptable carrier as described herein.

TABLE 1
Exemplary Compositions #1-17

| Ingredient | | | | | | | | | | | | | | | | | |
|---|---|---|---|---|---|---|---|---|---|---|---|---|---|---|---|---|---|
| (% by volume) | #1 | #2 | #3 | #4 | #5 | #6 | #7 | #8 | #9 | #10 | #11 | #12 | #13 | #14 | #15 | #16 | #17 |
| Minoxidil | 2.5 | — | 3 | 1.5 | — | 3 | 3.5 | 4 | 4.5 | 5 | 4.5 | 4 | 3.5 | 3 | 2.5 | 5 | — |
| Pyrrolidinyl | — | 2.5 | — | 1.5 | 3 | — | — | — | — | — | 0.5 | 1 | 1.5 | 2 | 2.5 | — | 5 |
| diaminopyrimidine | | | | | | | | | | | | | | | | | |
| oxide | | | | | | | | | | | | | | | | | |
| Melatonin | 1 | 1 | 1.5 | 1.5 | 1.5 | 1.5 | 1 | 1 | 1 | 1 | 1.5 | 1.5 | 1.5 | 1.5 | 1.5 | 1 | 1 |
| Ubiquinone | 0.25 | 0.5 | 0.25 | 0.5 | 0.25 | 0.5 | 0.25 | 0.5 | 0.25 | 0.4 | 0.5 | 0.25 | 0.5 | 0.25 | 0.5 | 0.25 | 0.5 |
| Finasteride | 0.5 | — | 0.25 | 0.25 | — | 0.25 | 0.25 | 0.25 | 0.25 | 0.25 | 0.25 | 0.25 | 0.25 | 0.25 | 0.25 | 0.5 | — |
| Emodin | — | 0.1 | 0.1 | 0.1 | 0.1 | 0.1 | 0.1 | 0.1 | 0.1 | 0.1 | 0.1 | 0.1 | 0.1 | 0.1 | 0.1 | — | 0.1 |
| Equol | — | 0.1 | 0.1 | 0.1 | 0.1 | 0.1 | 0.1 | 0.1 | — | | — | 0.1 | 0.1 | 0.1 | 0.1 | 0.1 | 0.1 |
| Genistein | 0.1 | — | 0.1 | 0.1 | 0.1 | 0.1 | 0.1 | — | 0.1 | 0.1 | 0.1 | — | 0.1 | 0.1 | 0.1 | 0.1 | 0.1 |
| Kaempferol | 0.1 | 0.1 | — | 0.1 | 0.1 | 0.1 | — | 0.1 | 0.1 | 0.1 | 0.1 | 0.1 | — | 0.1 | 0.1 | 0.1 | 0.1 |
| Caffeic acid | 0.1 | 0.1 | 0.1 | — | 0.1 | — | 0.1 | 0.1 | 0.1 | 0.1 | 0.1 | 0.1 | 0.1 | — | 0.1 | 0.1 | 0.1 |
| phenethyl ester | | | | | | | | | | | | | | | | | |
| Zinc oxide | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Allicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Eugenol | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Capsaicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Urea | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Vitamin C | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Vitamin E | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Beta-carotene | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| Copper peptide | 0.1 | — | 0.1 | — | 0.1 | — | 0.1 | — | 0.1 | — | 0.1 | — | 0.1 | — | 0.1 | — | 0.1 |
| Tyrosine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Arginine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| EGCG | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Clove oil | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 |

TABLE 2
Exemplary Compositions #18-32

| Ingredient | | | | | | | | | | | | | | | |
|---|---|---|---|---|---|---|---|---|---|---|---|---|---|---|---|
| (% by volume) | #18 | #19 | #20 | #21 | #22 | #23 | #24 | #25 | #26 | #27 | #28 | #29 | #30 | #31 | #32 |
| Pyrrolidinyl | 5 | 4.5 | 4 | 3.5 | 3 | 2.5 | 3 | 3.5 | 4 | 4.5 | 5 | 5 | 5 | 5 | 5 |
| diaminopyrimidine | | | | | | | | | | | | | | | |
| oxide | | | | | | | | | | | | | | | |
| Melatonin | 1 | 1 | 1 | 1 | 1 | 1 | 1 | 1 | 1 | 1 | 1.5 | 1.5 | 1.5 | 1.5 | 1.5 |
| Ubiquinone | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Emodin | — | 0.1 | 0.1 | 0.1 | 0.1 | 0.1 | 0.1 | 0.1 | 0.1 | 0.1 | 0.1 | 0.1 | 0.1 | 0.1 | 0.1 |
| Equol | — | 0.1 | 0.1 | 0.1 | 0.1 | 0.1 | 0.1 | 0.1 | — | | — | 0.1 | 0.1 | 0.1 | 0.1 |
| Genistein | 0.1 | — | 0.1 | 0.1 | 0.1 | 0.1 | 0.1 | — | 0.1 | 0.1 | 0.1 | — | 0.1 | 0.1 | 0.1 |
| Kaempferol | 0.1 | 0.1 | — | 0.1 | 0.1 | 0.1 | — | 0.1 | 0.1 | 0.1 | 0.1 | 0.1 | — | 0.1 | 0.1 |
| Caffeic acid | 0.1 | 0.1 | 0.1 | — | 0.1 | — | 0.1 | 0.1 | 0.1 | 0.1 | 0.1 | 0.1 | 0.1 | — | 0.1 |
| phenethyl ester | | | | | | | | | | | | | | | |
| Zinc oxide | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Allicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Eugenol | 0.5 | 0.75 | 1 | 1.25 | 1.5 | 1.75 | 0.5 | 0.5 | 0.5 | 0.5 | 0.5 | 0.75 | 1 | 1.25 | 1.5 |
| Capsaicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Urea | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Vitamin C | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Vitamin E | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Beta-carotene | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| Copper peptide | 0.1 | — | 0.1 | — | 0.1 | — | 0.1 | — | 0.1 | — | 0.1 | — | 0.1 | — | 0.1 |
| Tyrosine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Arginine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| EGCG | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Clove oil | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 |

TABLE 3
Exemplary Compositions #33-47

| Ingredient | | | | | | | | | | | | | | | |
|---|---|---|---|---|---|---|---|---|---|---|---|---|---|---|---|
| (% by volume) | #33 | #34 | #35 | #36 | #37 | #38 | #39 | #40 | #41 | #42 | #43 | #44 | #45 | #46 | #47 |
| Minoxidil | 2.5 | 2.75 | 3 | 3.25 | 3.5 | 3.75 | 4 | 4.25 | 4.50 | 4.75 | 5 | 5 | 5 | 5 | 5 |
| Pyrrolidinyl | 2.5 | 2.25 | 2 | 1.75 | 1.50 | 1.25 | 1 | 0.75 | 0.50 | 0.25 | 0.25 | — | — | — | — |
| diaminopyrimidine | | | | | | | | | | | | | | | |
| oxide | | | | | | | | | | | | | | | |
| Melatonin | 1.25 | 1.25 | 1.5 | 1.75 | 2 | 1.75 | 1.5 | 1.25 | 1 | 1 | 1.5 | 1.5 | 1.5 | 1.5 | 1.5 |
| Ubiquinone | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Finasteride | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Emodin | — | 0.1 | 0.1 | 0.1 | 0.1 | 0.1 | 0.1 | 0.1 | 0.1 | 0.1 | 0.1 | 0.1 | 0.1 | 0.1 | 0.1 |
| Equol | — | 0.1 | 0.1 | 0.1 | 0.1 | 0.1 | 0.1 | 0.1 | — | | — | 0.1 | 0.1 | 0.1 | 0.1 |
| Genistein | 0.1 | — | 0.1 | 0.1 | 0.1 | 0.1 | 0.1 | — | 0.1 | 0.1 | 0.1 | — | 0.1 | 0.1 | 0.1 |
| Kaempferol | 0.1 | 0.1 | — | 0.1 | 0.1 | 0.1 | — | 0.1 | 0.1 | 0.1 | 0.1 | 0.1 | — | 0.1 | 0.1 |
| Caffeic acid | 0.1 | 0.1 | 0.1 | — | 0.1 | — | 0.1 | 0.1 | 0.1 | 0.1 | 0.1 | 0.1 | 0.1 | — | 0.1 |
| phenethyl ester | | | | | | | | | | | | | | | |
| Zinc oxide | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Allicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Eugenol | 0.5 | 0.75 | 1 | 1.25 | 1.5 | 1.75 | 0.5 | 0.5 | 0.5 | 0.5 | 0.5 | 0.75 | 1 | 1.25 | 1.5 |
| Capsaicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Urea | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Vitamin C | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Vitamin E | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Beta-carotene | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| Copper peptide | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Tyrosine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Arginine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| EGCG | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Clove oil | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 |

TABLE 4
Exemplary Compositions #48-62

| Ingredient | | | | | | | | | | | | | | | |
|---|---|---|---|---|---|---|---|---|---|---|---|---|---|---|---|
| (% by volume) | #48 | #49 | #50 | #51 | #52 | #53 | #54 | #55 | #56 | #57 | #58 | #59 | #60 | #61 | #62 |
| Minoxidil | 2.5 | 2.75 | 3 | 3.25 | 3.5 | 3.75 | 4 | 4.25 | 4.50 | 4.75 | 5 | 5 | 5 | 5 | 5 |
| Pyrrolidinyl | 2.5 | 2.25 | 2 | 1.75 | 1.5 | 1.25 | 1 | 0.75 | 0.50 | 0.25 | 0.25 | — | — | — | — |
| diaminopyrimidine | | | | | | | | | | | | | | | |
| oxide | | | | | | | | | | | | | | | |
| Melatonin | 1.25 | 1.25 | 1.5 | 1.75 | 2 | 1.75 | 1.5 | 1.25 | 1 | 1 | 1.5 | 1.5 | 1.5 | 1.5 | 1.5 |
| Ubiquinone | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Finasteride | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Emodin | — | 0.1 | 0.1 | 0.1 | 0.1 | 0.1 | 0.1 | 0.1 | 0.1 | 0.1 | 0.1 | 0.1 | 0.1 | 0.1 | 0.1 |
| Equol | — | 0.1 | 0.1 | 0.1 | 0.1 | 0.1 | 0.1 | 0.1 | — | | — | 0.1 | 0.1 | 0.1 | 0.1 |
| Genistein | 0.1 | — | 0.1 | 0.1 | 0.1 | 0.1 | 0.1 | — | 0.1 | 0.1 | 0.1 | — | 0.1 | 0.1 | 0.1 |
| Kaempferol | 0.1 | 0.1 | — | 0.1 | 0.1 | 0.1 | — | 0.1 | 0.1 | 0.1 | 0.1 | 0.1 | — | 0.1 | 0.1 |
| Caffeic acid | 0.1 | 0.1 | 0.1 | — | 0.1 | — | 0.1 | 0.1 | 0.1 | 0.1 | 0.1 | 0.1 | 0.1 | — | 0.1 |
| phenethyl ester | | | | | | | | | | | | | | | |
| Zinc oxide | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Allicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Eugenol | 0.5 | 0.75 | 1 | 1.25 | 1.5 | 1.75 | 0.5 | 0.5 | 0.5 | 0.5 | 0.5 | 0.75 | 1 | 1.25 | 1.5 |
| Capsaicin | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Urea | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Vitamin C | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Vitamin E | 0.25 | 0.25 | 0.25 | 0.25 | 0.25 | 0.25 | 0.25 | 0.25 | 0.25 | 0.25 | 0.25 | 0.25 | 0.25 | 0.25 | 0.25 |
| Beta-carotene | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| Copper peptide | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Tyrosine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| Arginine | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| EGCG | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Clove oil | 0.15 | 0.15 | 0.15 | 0.15 | 0.15 | 0.15 | 0.15 | 0.15 | 0.15 | 0.15 | 0.15 | 0.15 | 0.15 | 0.15 | 0.15 |

TABLE 5
Exemplary Composition #63

| Ingredient | |
|---|---|
| (% by volume) | #63 |
| Minoxidil¹ | 0-3 |
| Pyrrolidinyl diaminopyrimidine oxide¹ | 0-3* |
| Finasteride¹ | 0-3* |
| Emodin² | 0-3^{#} |
| Equol² | 0-0.3^{#} |
| Genistein² | 0-0.3^{#} |
| Kaempferol² | 0-0.3^{#} |
| Caffeic acid phenethyl ester² | 0-0.3^{#} |
| Melatonin | 1-2 |
| Ubiquinone | 0.5-1 |
| Zinc oxide | 0.25 |
| Urea | 0.25 |
| Vitamin E | 0.1-0.25 |
| Beta-carotene | 0.01-0.05 |
| Arginine | 0.1 |
| Tyrosine | 0.1 |
| EGCG | 0.1-0.5 |
| Clove oil | 0.01-3 |
| St. John's wort oil | 0.05-3 |

¹minoxidil and/or pyrrolidinyl diaminopyrimidine oxide and finasteride must be included.
²at least two of the indicated ingredients must be included.

TABLE 6
Exemplary Composition #64

| Ingredient | |
|---|---|
| (% by volume) | #64 |
| Minoxidil¹ | 0-5* |
| Pyrrolidinyl diaminopyrimidine oxide¹ | 0-5* |
| Finasteride¹ | 0-5* |
| Emodin² | 0-3# |
| Equol² | 0-0.3# |
| Genistein² | 0-0.3# |
| Kaempferol² | 0-0.3# |
| Caffeic acid phenethyl ester² | 0-0.3# |
| Melatonin | 1-2 |
| Ubiquinone | 0.5-1 |
| Zinc oxide | 0.25 |
| Allicin | 0.1 |
| Capcaicin | 0.1 |
| Urea | 0.25 |
| Vitamin C | 0.1 |
| Vitamin E | 0.1-0.25 |
| Beta-carotene | 0.01-05 |
| Arginine | 0.1 |
| EGCG | 0.1-0.5 |
| Clove oil | 0.01-3 |
| St. John's wort oil | 0.05-3 |

¹minoxidil and/or pyrrolidinyl diaminopyrimidine oxide and finasteride must be included.
²at least two of the indicated ingredients must be included.

### Methods of Manufacture

In one aspect, provided herein are processes for making the compositions provided herein. The compositions provided herein can generally be made by methods known in the art, in view of the following guidance.

In general, the compositions are made by preparing an oil phase, an aqueous phase, and then emulsify the two phases until a desired homogeneity is obtained.

In some embodiments, the oil phase may be prepared by mixing at least two oils selected from a group of olive oil, rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, and hazelnut oil. In some embodiments, the oil phase may be prepared by olive oil with at least one oil selected from a group of rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, or hazelnut oil.

Once the oils are mixed, then at least one emulsifier, such as cetyl alcohol, is added and the resulting mixture is mixed until a desired homogeneity is obtained. In some embodiments, the mixture is mixed for at least 30 minutes. In some embodiments, the mixture is heated while mixed. In some embodiments, the mixture is heated to about 60-65° C. In some embodiments, the mixture is mixed for about 30 minutes at about 60-65° C. Then, the active ingredients that are soluble in oil phase, such as ubiquinone, melatonin, minoxidil, and finasteride, are dissolved in the oil mixture.

Once the oil phase-soluble active ingredients are dissolved in the oil mixture, then the organic solvent and the polyol are added to the oil mixture in addition to more of an emulsifier, such as polysorbates 20. The resulting mixture is mixed for at least ten minutes.

In some embodiments, the aqueous phase may be prepared by mixing water with any active ingredients that are soluble in water, such as zinc oxide, urea, vitamin C, and vitamin B complex (vitamin B1, vitamin B2, vitamin B3, vitamin B5, vitamin B6, vitamin B7, vitamin B9, and vitamin B12).

In some embodiments, the water is distilled water. In some embodiments, 2.5 grams (g) of zinc oxide, 5 g of urea, 5 g of vitamin C, 0.1 g of vitamin B complex, and 1 liter of distilled water are mixed. In some embodiments, the mixture is mixed for at least 30 minutes. In some embodiments, the mixture is mixed at room temperature. In some embodiments, the mixture is mixed for at least 30 minutes at room temperature.

The aqueous phase is then added to the oil phase and mixed for at least 5 minutes. Then, the preservative and optionally vitamin E are added. The resulting mixture is then continued to be mixed in a mechanical or ultrasonic homogenizator until a desired homogeneity is obtained. Inactive ingredients such as anti-microbials, preservatives, and pH regulators are added either immediately prior to or after mixing in a homogenizator.

The compositions provided herein may be formulated for topical administration. In some embodiments, the compositions provided herein may be formulated into ointment, cream, suspension, lotion, powder, solution, paste, gel, spray, aerosol, foam, or oil. In some embodiments, the compositions provided herein may be formulated into a cream, ointment, gel, or foam.

The instant compositions may also include, for example, micelles or liposomes, or some other encapsulated form, or may be administered in an extended release form to provide a prolonged storage and/or delivery effect.

### Methods of Use

In one aspect, provided herein is a method of growing hair or stimulating hair growth comprising administering to a subject in need thereof a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof; and a cosmetically effective amount of one or more of an epigenetic modifier. The coenzyme and epigenetic modifier may be part of a composition, and may optionally include a cosmetically acceptable carrier and/or one or more antioxidants (of any embodiment described herein). Prior to, concurrent with, or after such administration, the method may further include administering a cosmetically effective amount of one or more of a blood circulator (of any embodiment described herein) and a 5-alpha reductase modulator (of any embodiment described herein), optionally within about 0-2 hours from administration of the coenzyme and epigenetic modifier.

In some embodiments, the administering is carried out topically on a skin of the subject. In some embodiments, the skin comprises the subject's scalp or a portion thereof.

In some embodiments, the hair is an eyebrow hair. In some embodiments, the hair is an eyelash hair.

In some embodiments, the hair is an eyebrow hair and the method further comprises administering St. John's wort oil. In some embodiments, the hair is an eyelash hair and the method further comprises administering St. John's wort oil.

A unit dose of any composition described herein can be administered in a variety of dosing schedules, depending on the judgment of the clinician, needs of the subject, and so forth. The specific dosing schedule will be known by those of ordinary skill in the art or can be determined experimentally using routine methods.

Depending upon the dosage amount and precise hair loss condition to be treated, the compositions described herein is administered once a day, twice a day, three times a day, four times a day, or more. In some embodiments, the compositions described herein are administered twice a day. In some embodiments, the compositions described herein are administered in the morning and in the evening. In some embodiments, the compositions described herein are administered early in the morning between about 7 am to about 9 am. In some embodiments, the compositions described herein are administered about 1-2 hours or about 1.5-2 hours before going to bed. In some embodiments, the compositions described herein are administered early in the morning between about 7 am to about 9 am and about 1.5-2 hours before going to bed. In some embodiments, the compositions described herein are administered early in the morning between about 7 am to about 9 am and about immediately just before going to bed.

In some embodiments, the compositions described herein are administered one, two, three, or four times daily for one week, two weeks, three weeks, four weeks, five weeks, six weeks, seven weeks, eight weeks, or more. In some embodiments, the compositions described herein are administered for at least one month. In some embodiments, the compositions described herein are administered for at least six weeks, seven weeks, or eight weeks.

In some embodiments, the method comprises administering any of the compositions described herein for at least 7 days, at least 14 days, at least 21 days, at least 28 days, at least 35 days, at least 40 days, at least 45 days, at least 50 days, at least 55 days, at least 60 days, or more. In some embodiments, any of the compositions described herein is administered for at least 45 days.

In some embodiments, the method comprises administering any of the compositions described herein twice a day for at least 7 days, at least 14 days, at least 21 days, at least 28 days, at least 35 days, at least 40 days, at least 45 days, at least 50 days, at least 55 days, at least 60 days, or more. In some embodiments, any of the compositions described herein is administered twice a day for at least 45 days.

Cosmetic amounts can be empirically determined and will vary with the particular condition being treated, the subject, and the efficacy and toxicity of each of the active agents contained in the compositions described herein. The actual dose to be administered will vary depending upon the age, weight, and general condition of the subject as well as the severity of the condition being treated, the judgment of the health care professional, and particular combination being administered.

Cosmetically effective amounts can be determined by those skilled in the art, and will be adjusted to the requirements of each particular case. Generally, a cosmetically effective amount of any of the compositions disclosed herein will range from a total daily dosage of about 1 mL to about 5 mL, preferably from about 2 mL to about 3 mL. The total daily dosage may be administered via two or more applications. For example, if the total daily dosage is about 2 mL to about 3 mL, about 0.5-1 mL may be applied in the morning and about 1-2 mL may be applied in the evening.

In some embodiments, the cosmetically effective amounts can vary depending on the size of the hair loss area. In some embodiments, any of the compositions disclosed herein may be administered in an amount sufficient to evenly cover the hair loss area. In some embodiments, about 1-5 mL of any of the compositions disclosed herein is administered to a subject in need thereof. In some embodiments, about 1-4 mL, or about 1-3 mL, or about 1-2 mL of any of the compositions disclosed herein is administered to a subject in need thereof. In some embodiments, about 1-2 mL of any of the compositions disclosed herein is administered.

### Methods of Administration of the Cosmetic Compositions of the Present Technology

In one aspect, provided herein is a method of growing hair or stimulating hair growth comprising administering to a subject in need thereof a cosmetically effective amount of ubiquinone or a cosmetically acceptable salt thereof and a cosmetically effective amount of an epigenetic modifier as described herein.

In some embodiments, the administering is carried out topically on a skin of the subject. In some embodiments, the skin comprises the subject's scalp or a portion thereof.

In some embodiments, the hair is an eyebrow hair. In some embodiments, the hair is an eyelash hair.

In some embodiments, the hair is an eyebrow hair and the method further comprises administering St. John's wort oil. In some embodiments, the hair is an eyelash hair and the method further comprises administering St. John's wort oil.

Depending upon the dosage amount and precise hair loss condition to be treated, the cosmetically effective amount of ubiquinone or the cosmetically acceptable salt thereof and the cosmetically effective amount of the epigenetic modifier are administered once a day, twice a day, three times a day, four times a day, or more. In some embodiments, the cosmetically effective amount of ubiquinone or the cosmetically acceptable salt thereof and the cosmetically effective amount of the epigenetic modifier are administered twice a day.

In some embodiments, the cosmetically effective amount of ubiquinone or the cosmetically acceptable salt thereof and the cosmetically effective amount of the epigenetic modifier are administered one, two, three, or four times daily for one week, two weeks, three weeks, four weeks, five weeks, six weeks, seven weeks, eight weeks, or more. In some embodiments, the cosmetically effective amount of ubiquinone or the cosmetically acceptable salt thereof and the cosmetically effective amount of the epigenetic modifier are administered for at least one month. In some embodiments, the cosmetically effective amount of ubiquinone or the cosmetically acceptable salt thereof and the cosmetically effective amount of the epigenetic modifier are administered for at least six weeks, seven weeks, or eight weeks.

In some embodiments, the method comprises administering the cosmetically effective amount of ubiquinone or the cosmetically acceptable salt thereof and the cosmetically effective amount of the epigenetic modifier for at least 7 days, at least 14 days, at least 21 days, at least 28 days, at least 35 days, at least 40 days, at least 45 days, at least 50 days, at least 55 days, at least 60 days, or more. In some embodiments, the cosmetically effective amount of ubiquinone or the cosmetically acceptable salt thereof and the cosmetically effective amount of the epigenetic modifier are administered for at least 45 days.

In some embodiments, the method comprises administering the cosmetically effective amount of ubiquinone or the cosmetically acceptable salt thereof and the cosmetically effective amount of the epigenetic modifier twice a day for at least 7 days, at least 14 days, at least 21 days, at least 28 days, at least 35 days, at least 40 days, at least 45 days, at least 50 days, at least 55 days, at least 60 days, or more. In some embodiments, the cosmetically effective amount of ubiquinone or the cosmetically acceptable salt thereof and the cosmetically effective amount of the epigenetic modifier are administered twice a day for at least 45 days.

In some embodiments, the method further comprises administering to the subject a cosmetically effective amount of one or more terminal antioxidants or suicidal antioxidants as described herein.

In some embodiments, the method further comprises administering to the subject a cosmetically effective amount of a moisturizer as described herein.

In some embodiments, the method further comprises administering to the subject a cosmetically effective amount of an anti-fungal agent as described herein.

In some embodiments, the method further comprises administering to the subject a cosmetically effective amount of a UV filter as described herein.

In some embodiments, the method further comprises administering to the subject a cosmetically effective amount of a piloerector muscle relaxer as described herein.

In some embodiments, the method further comprises administering to the subject a cosmetically effective amount of a blood circulator as described herein.

In some embodiments, the method further comprises administering to the subject a cosmetically effective amount of a 5-alpha reductase modulator as described herein.

In some embodiments, the method further comprises administering to the subject a cosmetically effective amount of a non-terminal antioxidant or non-suicidal antioxidant as described herein.

In some embodiments, the method further comprises administering to the subject a cosmetically effective amount of one or more of a group of copper peptide, vitamin B1, vitamin B2, vitamin B3, vitamin B5, vitamin B6, vitamin B7, vitamin B9, vitamin B12, bimatoprost, tafluprost, travopost, latanoprost, caffeine, clove oil, and St. John's wort oil.

In some embodiments, the method further comprises administering to the subject a cosmetically effective amount of copper peptide. In some embodiments, the method further comprises administering to the subject a cosmetically effective amount of vitamin B1. In some embodiments, the method further comprises administering to the subject a cosmetically effective amount of vitamin B2. In some embodiments, the method further comprises administering to the subject a cosmetically effective amount of vitamin B3. In some embodiments, the method further comprises administering to the subject a cosmetically effective amount of vitamin B5. In some embodiments, the method further comprises administering to the subject a cosmetically effective amount of vitamin B6. In some embodiments, the method further comprises administering to the subject a cosmetically effective amount of vitamin B7. In some embodiments, the method further comprises administering to the subject a cosmetically effective amount of vitamin B9. In some embodiments, the method further comprises administering to the subject a cosmetically effective amount of vitamin B12. In some embodiments, the method further comprises administering to the subject a cosmetically effective amount of bimatoprost. In some embodiments, the method further comprises administering to the subject a cosmetically effective amount of tafluprost. In some embodiments, the method further comprises administering to the subject a cosmetically effective amount of travopost. In some embodiments, the method further comprises administering to the subject a cosmetically effective amount of latanoprost. In some embodiments, the method further comprises administering to the subject a cosmetically effective amount of caffeine. In some embodiments, the method further comprises administering to the subject a cosmetically effective amount of clove oil. In some embodiments, the method further comprises administering to the subject a cosmetically effective amount of St. John's wort oil.

Cosmetic amounts can be empirically determined and will vary with the particular condition being treated, the subject, and the efficacy and toxicity of each of the active agents described herein. The actual dose to be administered will vary depending upon the age, weight, and general condition of the subject as well as the severity of the condition being treated, the judgment of the health care professional, and particular combination being administered.

The active agents described herein may be administered simultaneously or separately with each other or with the cosmetically effective amount of ubiquinone and the cosmetically effective amount of the epigenetic modifier.

The present disclosure, thus generally described, will be understood more readily by reference to the following examples, which are provided by way of illustration and are not intended to be limiting of the present disclosure.

### EXAMPLES

### Example 1

### Preparation of Composition A

To prepare the oil phase, 1.5 liters (L) of olive oil, 0.1 L clove oil, 0.2 L of St. John's wort oil, 0.1 L of Rosemary oil, 0.1 L of pumpkin seed oil, 40 g cetyl alcohol, and 40 g cetearyl alcohol were mixed for 20 minutes. 250 g minoxidil, 150 g melatonin, 50 g ubiquinone, and 25 g finasteride were then added to the oil mixture. The oil mixture was then homogenized (via a homogenizator at about 500-1000 rpm) for at least 30 minutes at 60-65° C.

1.5 L glycerin, 1.5 L mono/dipropyleneglycol, and 1 L of polysorbates 20 were then added to the homogenized oil mixture and mixed for at least 10 minutes.

To prepare the aqueous phase, 10 g zinc oxide, 40 g urea, 20 g vitamin C, and 1.0 g vitamin B complex were added to 4 L of distilled water. The resulting mixture was mixed via a homogenizator at about 500-1000 rpm for at least 30 minutes at room temperature to prepare the aqueous phase.

The aqueous phase was then added to the oil phase and the resulting emulsion was mixed for at least 30 minutes. 10 g potassium sorbate and 10 mL vitamin E were then added. The resulting mixture was mixed via a homogenizator at about 500-1000 rpm for at least 30 minutes, whereupon final pH adjustment provided Composition A.

### Example 2

### Hair Growth Stimulation

10 individuals were provided with a composition with the following components: 85 mL olive oil, 5 mL clove oil, 5 mL rosemary oil, 5 mL St. John's wort oil, 250 mL glycerin, 250 mL mono/dipropyleneglycol, 300 ml distilled water, 100 mL polysorbate 20, 5 g cetyl alcohol, 2.5% minoxidil, 2.5% pyrrolidinyl diaminopyrimidine oxide, 1% melatonin, 0.5% ubiquinone, 0.5% finasteride, 0.25% zinc oxide, 0.5% vitamin C, 1% urea, 0.1% arginine, 0.5% caffeine, 0.5% epigallocatechine gallate (EGCG), 0.1% vitamin B varieties, 1% potassium sorbate, and 0.5% vitamin E. The individuals were instructed to agitate the composition thoroughly before each use.

The individuals were instructed to apply 1-2 mL of the composition on the scalp about 1.5-2 hours prior to sleeping and spread the composition equally (such as with a comb or hairbrush). The individuals were further instructed to apply 0.5-1 mL of the composition upon waking for the day and allow the composition to remain on the scalp for about 2-4 hours.

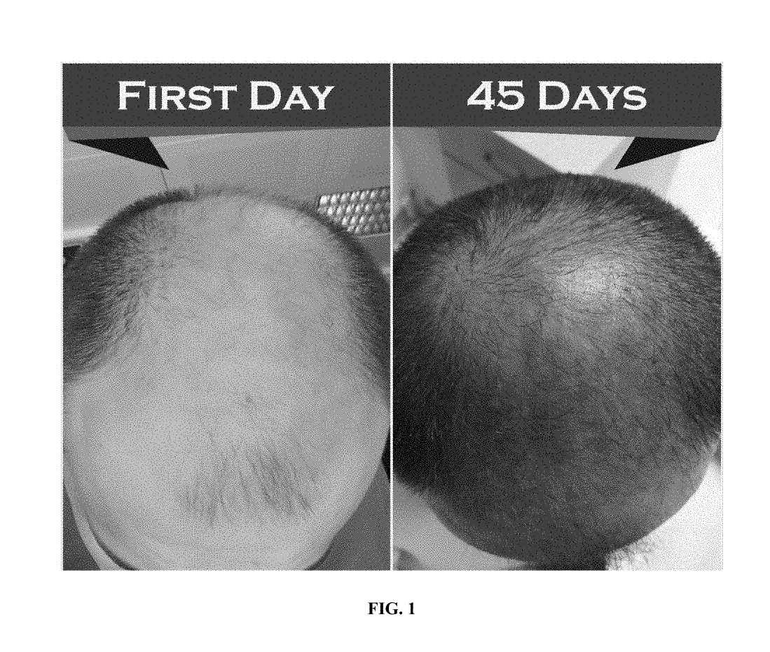

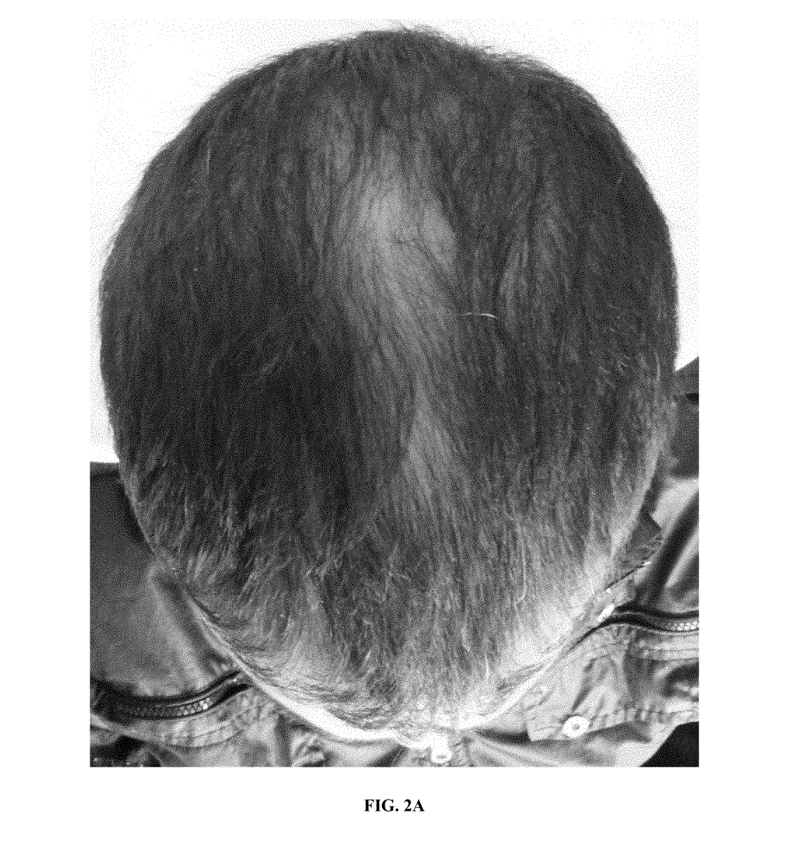

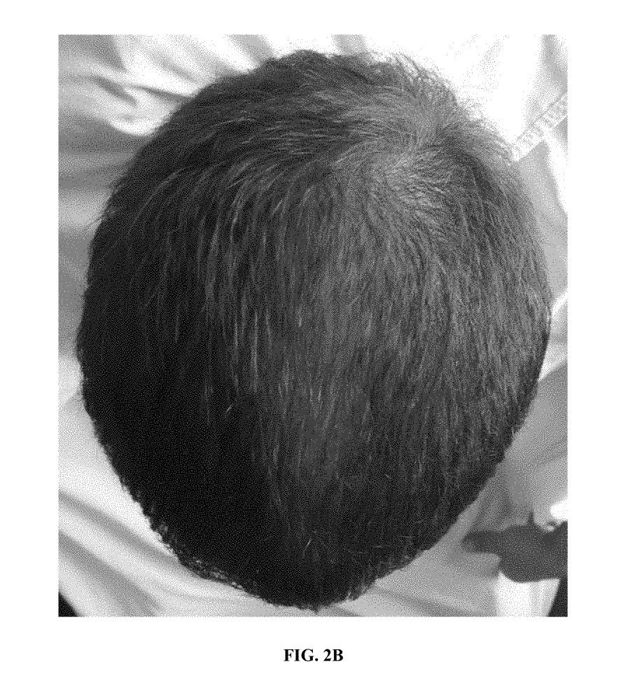

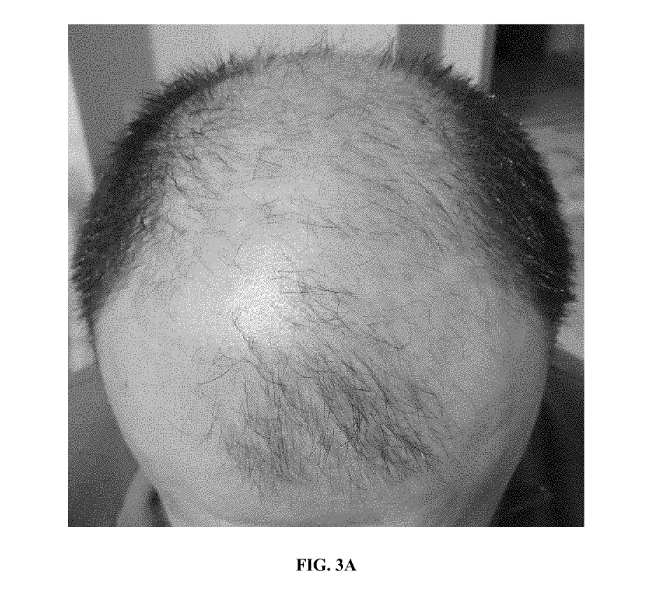

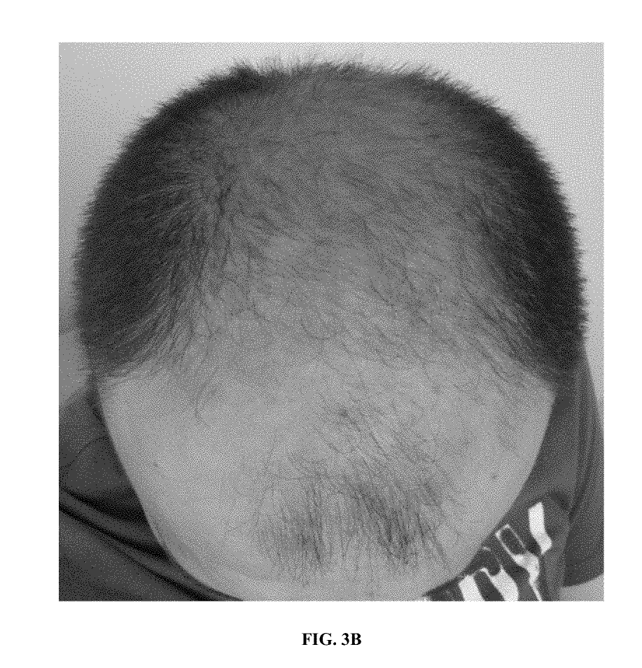

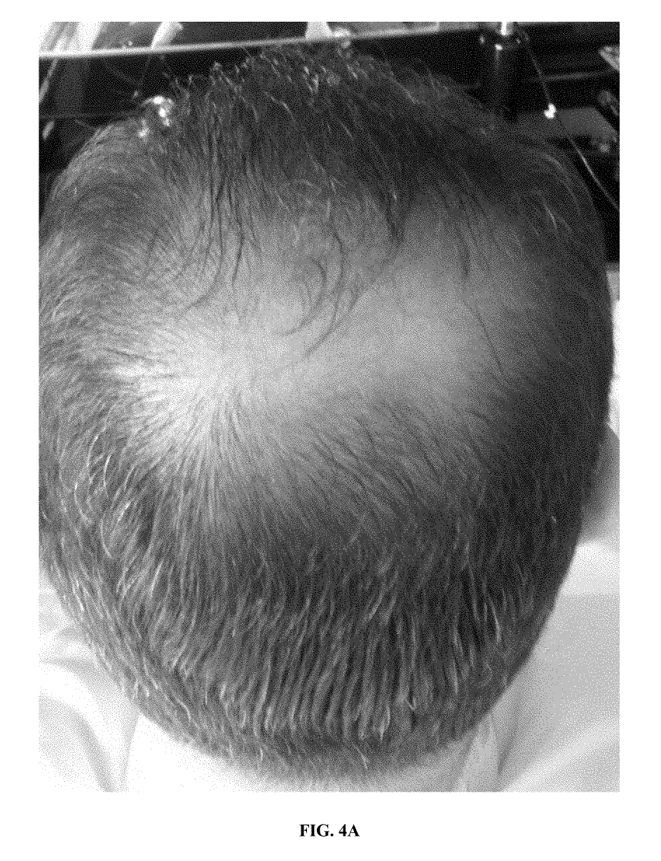

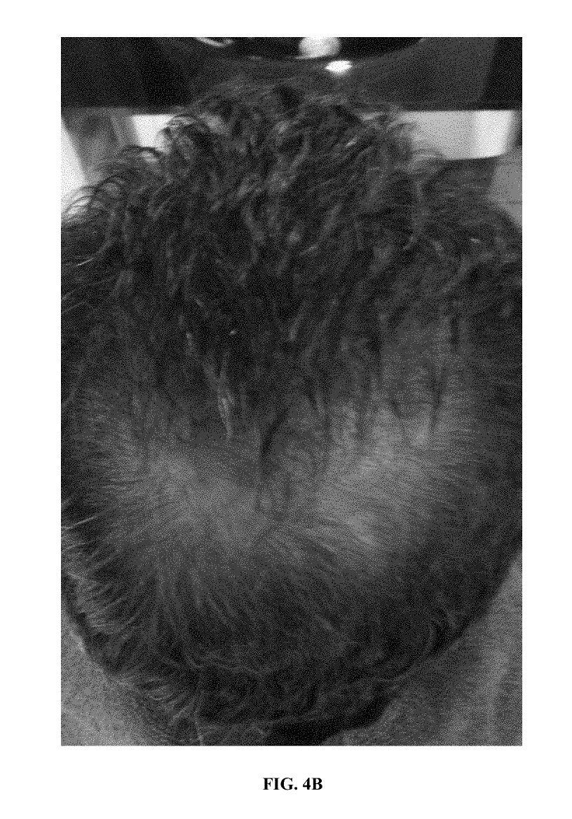

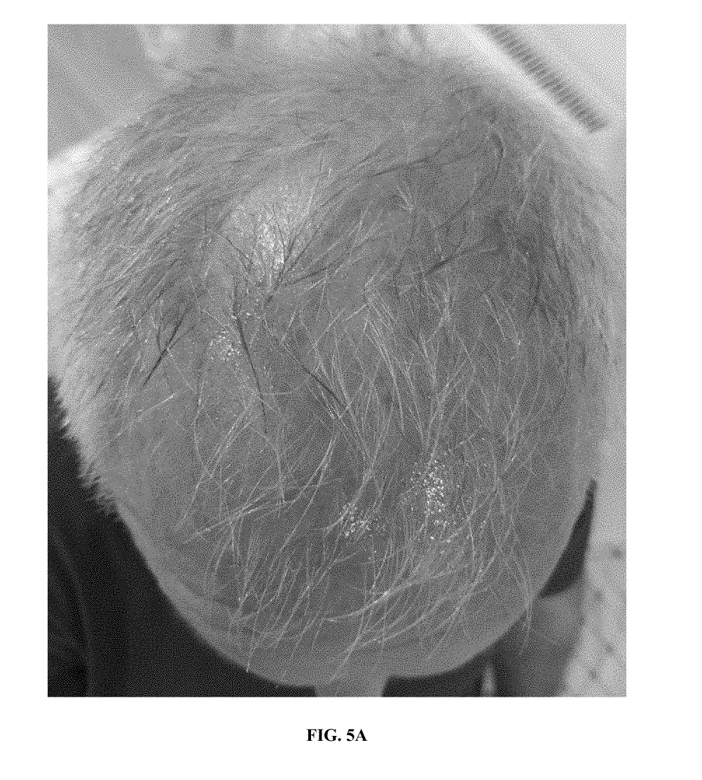

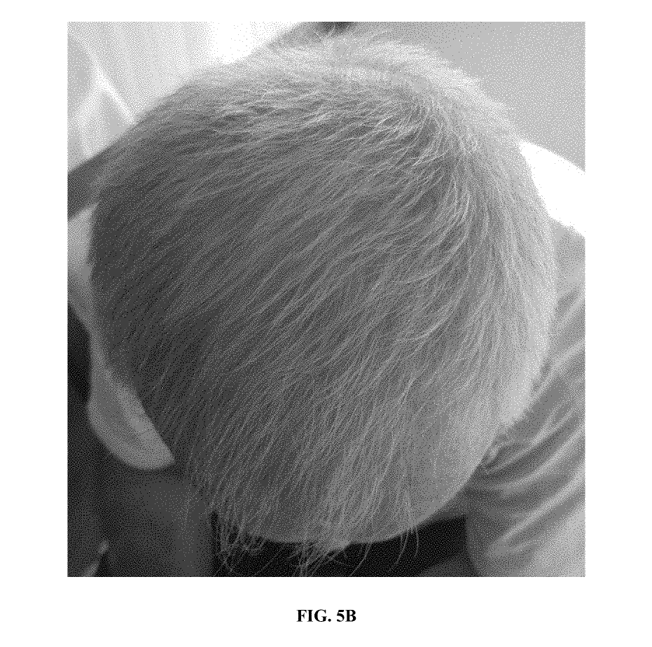

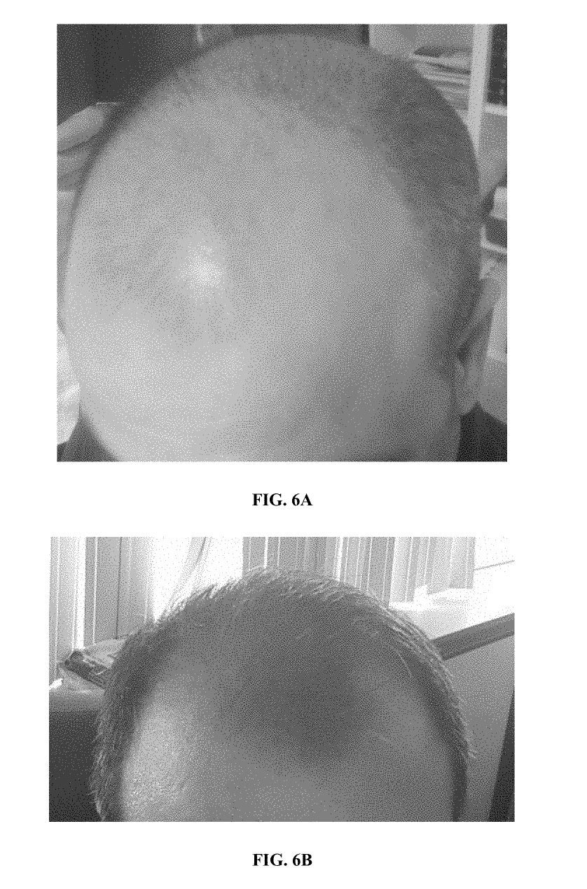

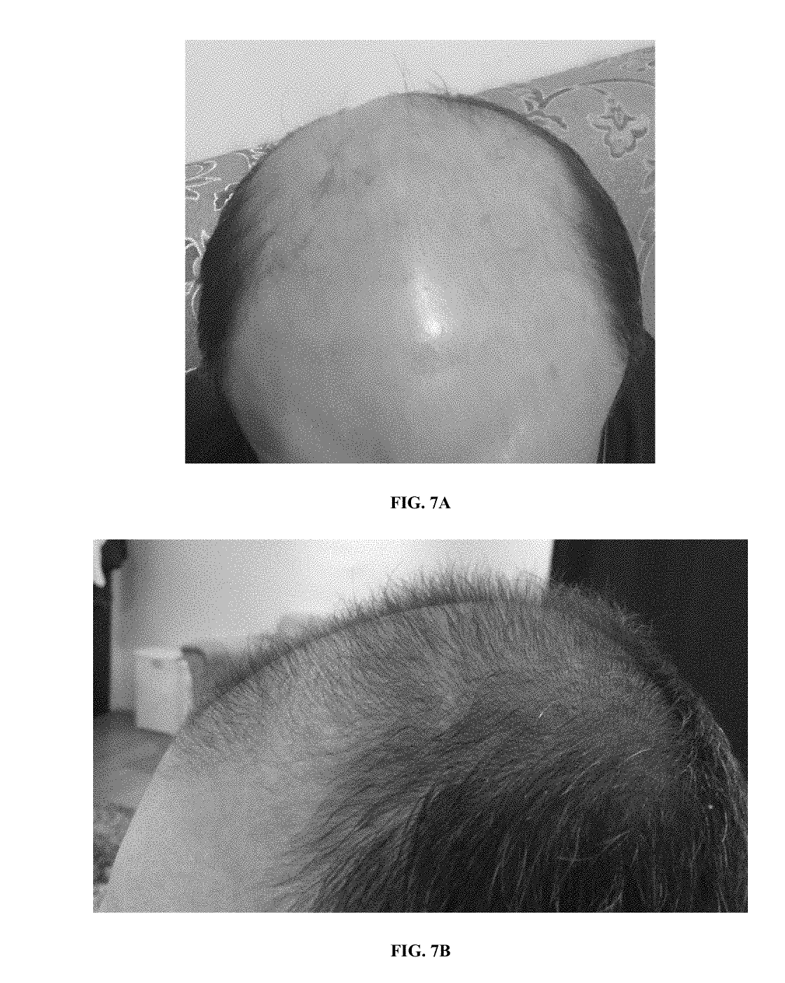

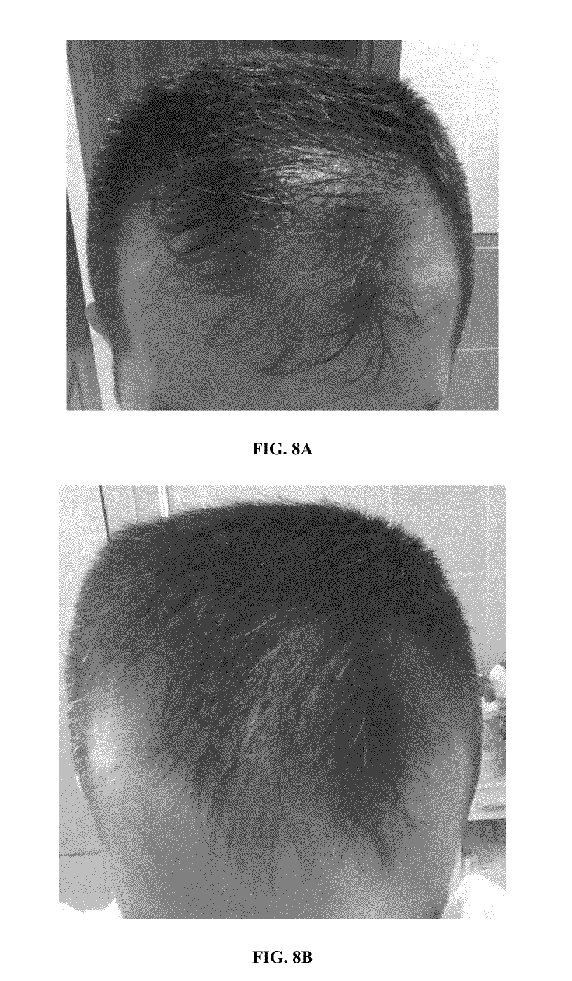

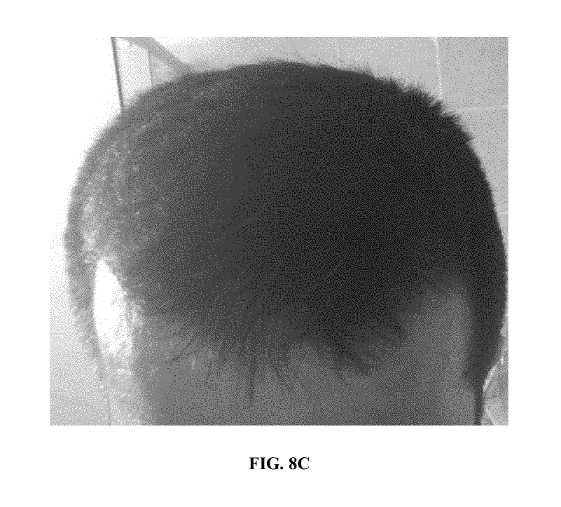

Significant results provided by such application are visually apparent on the individuals. The before and after pictures of the scalp of one male individual (taken by the individual) who administered the composition for 45 days are provided in FIG. 1. Before and after pictures of seven additional male individuals are provided in FIGS. 2A-B (45 days administration), 3A-B (45 days administration), 4A-B (90 days administration), 5A-B (30 days administration), 6A-B (120 days administration), 7A-B (84 days administration, and 8A-C (FIG. 8B=after 90 days administration; FIG. 8C=after 6 months administration).

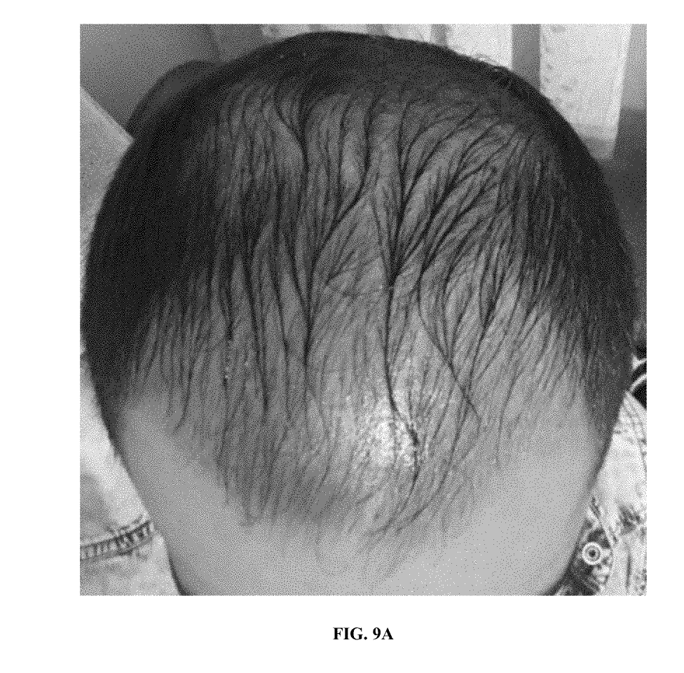

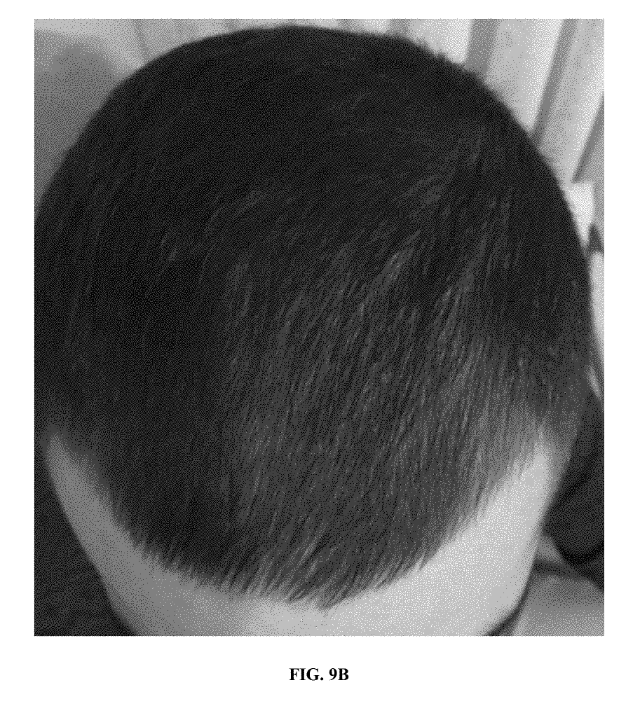

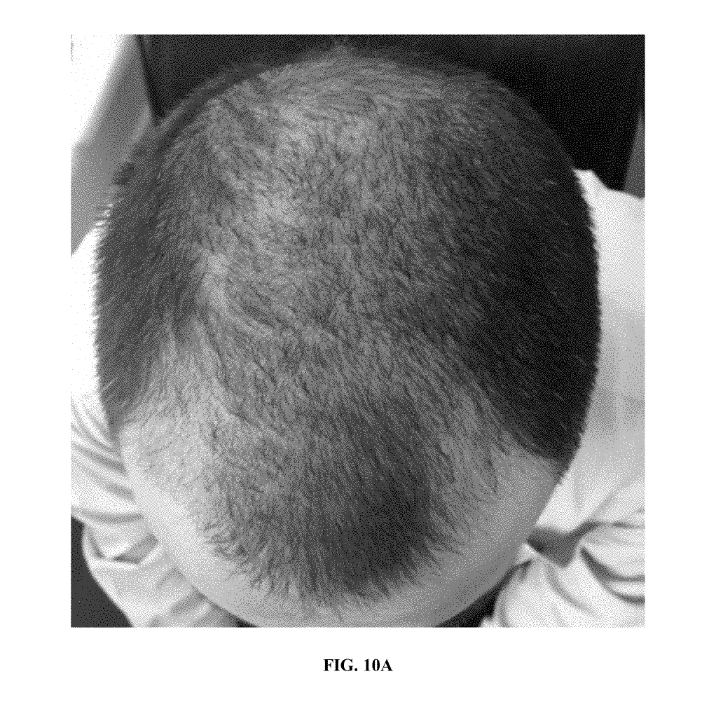

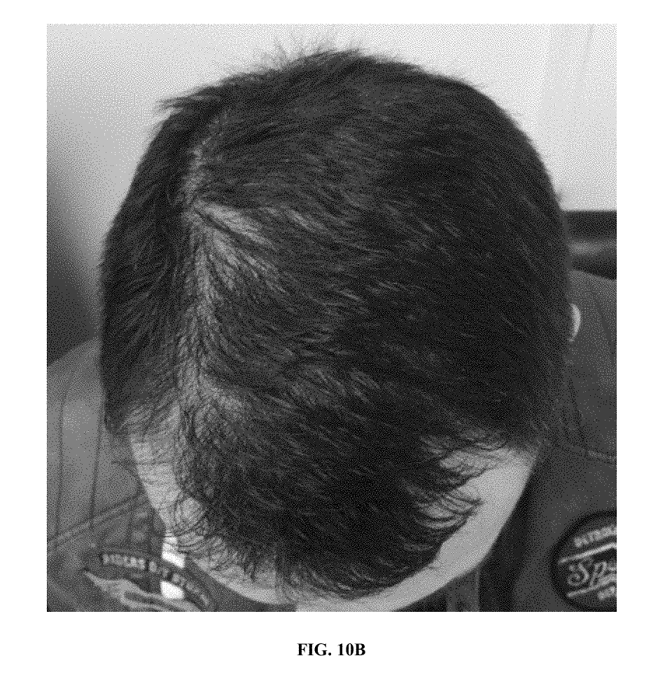

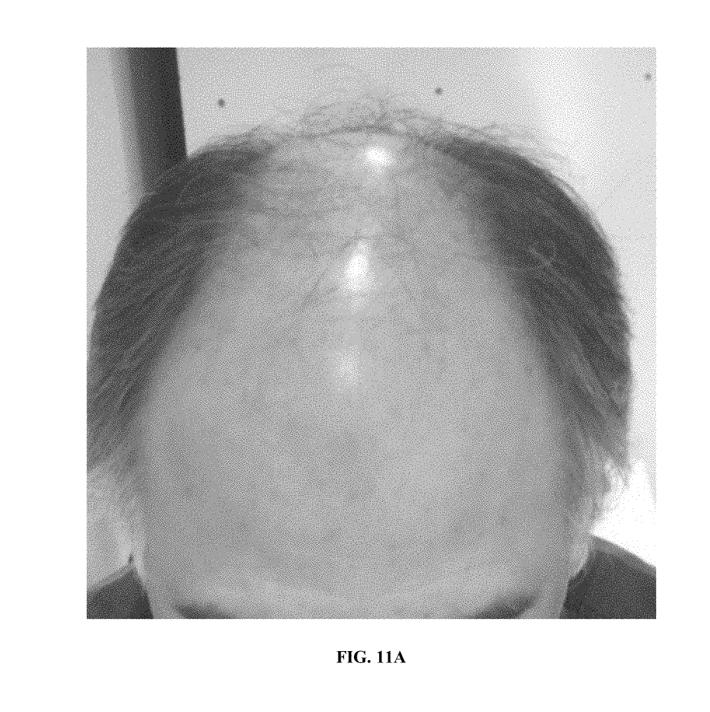

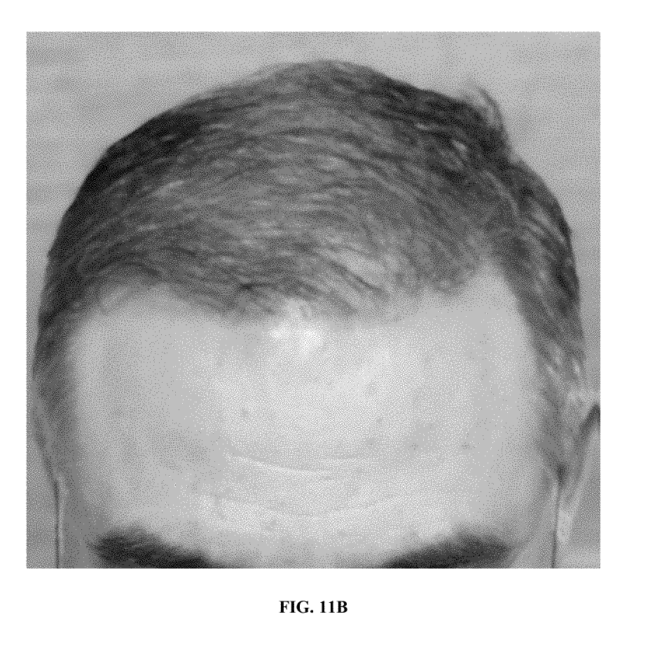

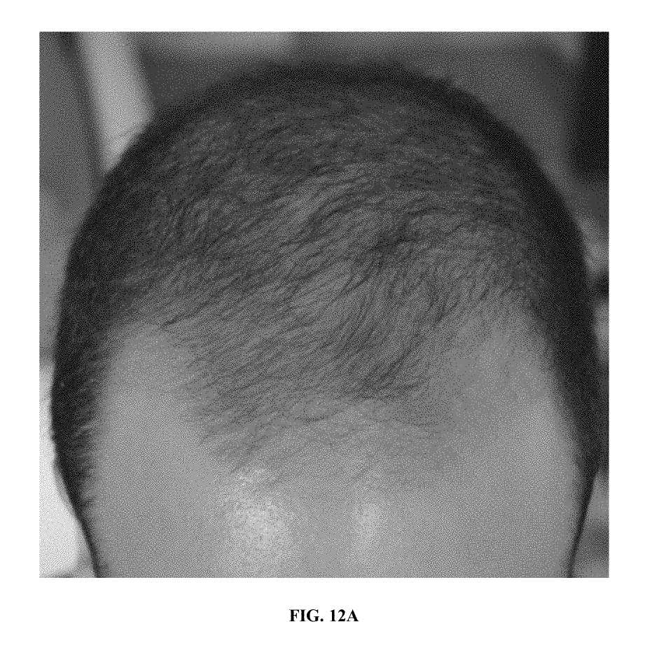

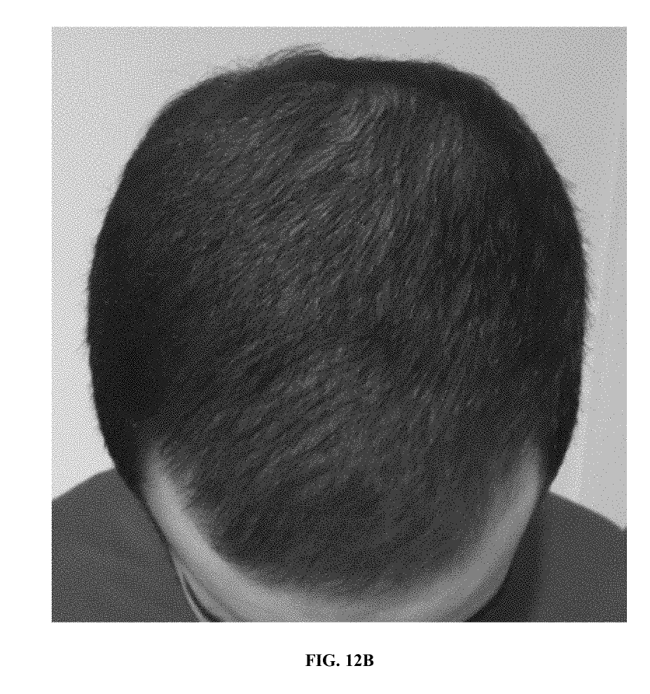

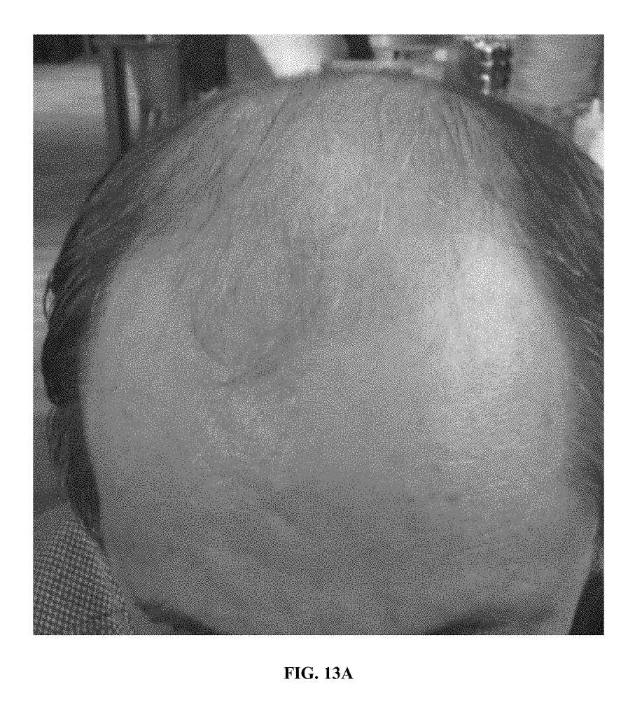

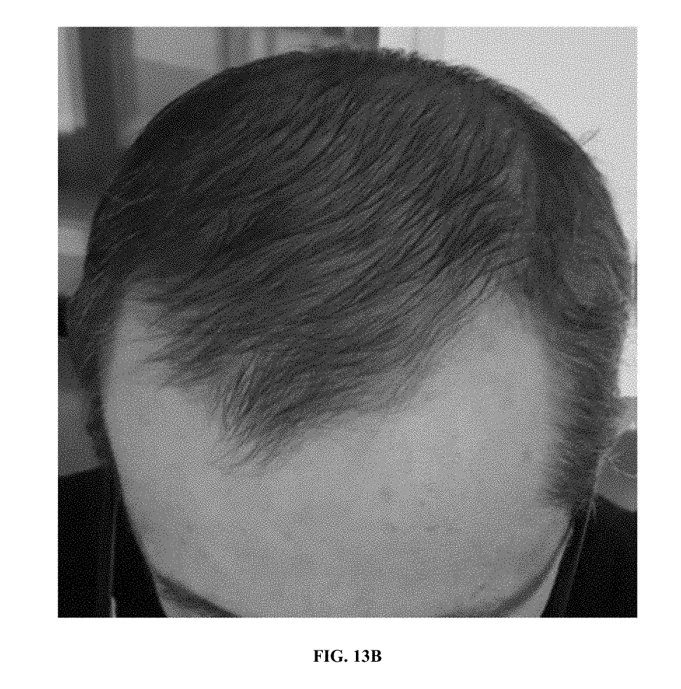

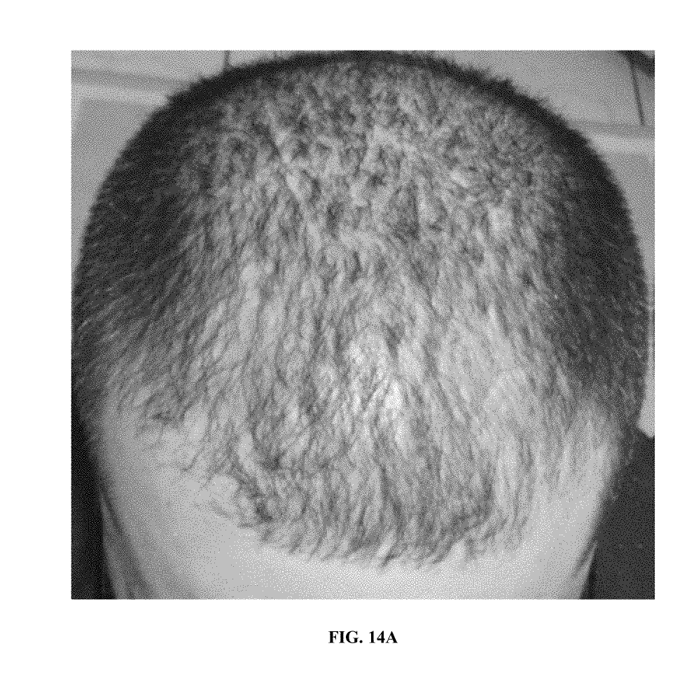

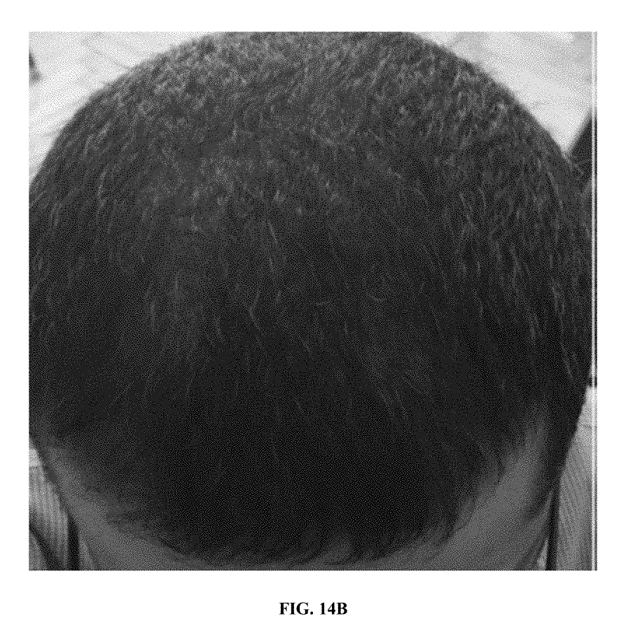

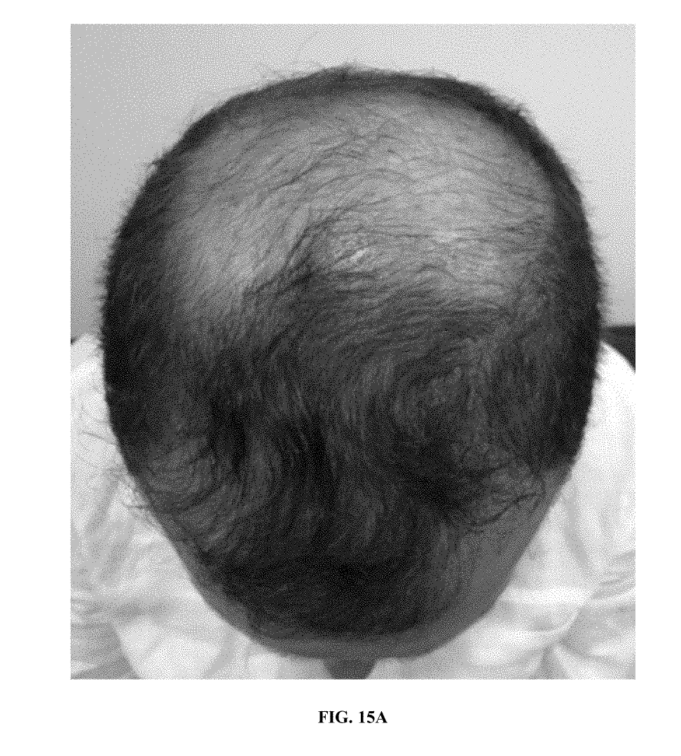

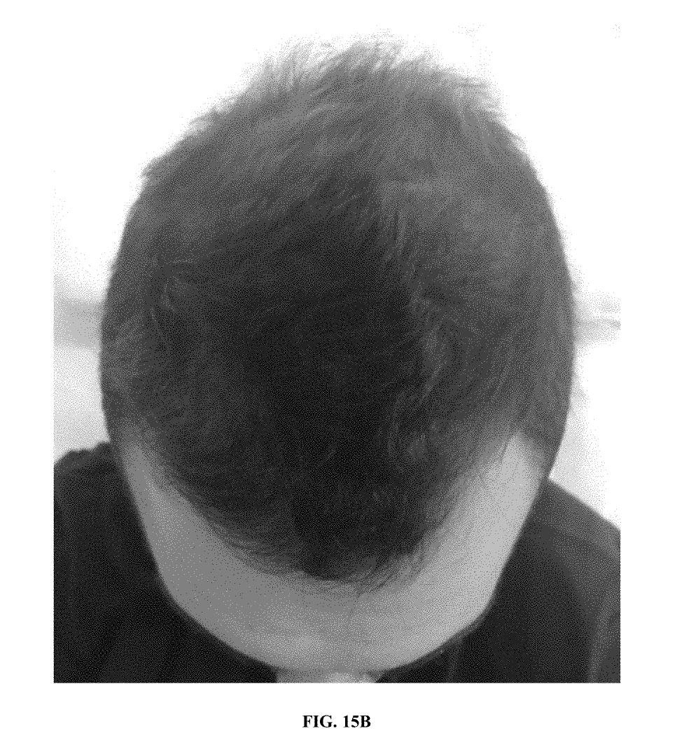

Further representative before and after pictures of individuals who administered the composition for 3 months are provided in FIGS. 9A-B, 10A-B, 11A-B, 12A-B, 13A-B, 14A-B, and 15A-B. Similar significant results were observed for females. In all of the numerous individuals undergoing administration of compositions of the present technology, severe adverse effects were not observed. Local itching and redness was observed in two individuals in the first two days of administration, for which antihistamines readily alleviated such symptoms; after about two days, the local itching and redness subsided and antihistamines were discontinued. One individual in one instance suffered orthostatic hypotension just after getting out of bed in the morning, however it is unclear if this was due to the composition or other health factors of that particular individual.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A composition comprising:
a) a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof;
b) a cosmetically effective amount of one or more of an epigenetic modifier;
c) a cosmetically effective amount of a blood circulator;
d) a cosmetically effective amount of a 5-alpha reductase modulator; and
e) a cosmetically acceptable carrier.

2. The composition of claim 1, wherein the composition comprises
c) about 0.05% to about 6.0% of the blood circulator by volume of the composition; and
d) about 0.05% to about 6.0% of the 5-alpha reductase modulator by volume of the composition.

3. The composition of claim 1, wherein the composition is formulated for administration to a female subject and comprises about 1.0% to about 6.0% by volume of the 5-alpha reductase modulator.

4. The composition of claim 3, wherein the composition further comprises about 0.5% to about 1.0% curcumin by volume of the composition.

5. The composition of claim 1, wherein the composition is formulated for administration to a male subject and comprises about 0.1% to about 0.6% by volume of the 5-alpha reductase modulator.

6. The composition of claim 1, wherein the cosmetically acceptable carrier comprising at least one of an organic solvent, a polyol, water, and an oil.

7. The composition of claim 1, wherein the epigenetic modifier comprises one or more members of the group consisting of melatonin, epigallocatechine gallate (EGCG), and valproic acid.

8. The composition of claim 1, wherein the composition further comprises one or more members selected from the group consisting of a moisturizer, an anti-fungal agent, an ultra-violet light filter (UV filter), and a piloerector muscle relaxer.

9. The composition of claim 8, wherein the moisturizer comprises urea, dexpanthenol (vitamin B5), or both.

10. The composition of claim 8, wherein the anti-fungal agent comprises allicin, eugenol, or both.

11. The composition of claim 8, wherein the UV filter comprises one or more members of the group consisting of zinc oxide, allicin, and beta-carotene.

12. The composition of claim 8, wherein the piloerector muscle relaxer comprises prazosin, melatonin, or both.

13. The composition of claim 1, wherein the composition further comprises an antioxidant, wherein the antioxidant comprises melatonin, 5-metoxytriptamine, cyclic 3-hydroxymelatonin, N-acetyl-5-methoxykynuramine, or a combination of any two or more thereof.

14. The composition of claim 2, wherein the blood circulator comprises one or more members of the group consisting of minoxidil, pyrrolidinyl diaminopyrimidine oxide, arginine, lidocaine, capsaicin, and curcumin.

15. The composition of claim 2, wherein the 5-alpha reductase modulator comprises one or more members of the group consisting of emodin, finasteride, eugenol, dutasteride, enoxolone, equol, beta-sitosterol, and genistein.

16. The composition of claim 1, wherein the composition comprises about 1% to about 35% oil by volume of the composition, wherein the oil comprises one or more of olive oil, rosemary oil, sweet almond oil, nigella sativa oil, St. John's wort oil, argan oil, theme oil, pumpkin seed oil, black marble oil, clove oil, red palm oil, horse tail oil, coconut oil, walnut oil, pine oil, nettle seed oil, castor oil, or hazelnut oil.

17. The composition of claim 1, wherein the composition comprises about 1% to about 10% oil by volume of the composition, wherein the oil comprises one or more of olive oil, clove oil, and St. John's wort oil.

18. A method of growing hair and/or stimulating hair growth comprising administering to a subject in need thereof
a cosmetically effective amount of a coenzyme that is one or more of the group selected from ubiquinone, Coenzyme A, Coenzyme Q2, Coenzyme Q4, Coenzyme Q9, Ubiquinol, Coenzyme Q1, plastoquinone, plastoquinol, and cosmetically acceptable salts of any one or more thereof; and
a cosmetically effective amount of one or more of an epigenetic modifier.

19. The method of claim 18, wherein the method further comprises administering a cosmetically effective amount of a blood circulator.

20. The method of claim 19, wherein the blood circulator is administered prior to or concurrent with administration of the coenzyme and epigenetic modifier.

21. The method of claim 18, wherein the composition is administered at least twice a day for at least 45 days.

22. The method of claim 18, wherein the administering comprises topical administration on skin of the subject.

23. The method of claim 22, wherein the skin comprises at least a portion of scalp of the subject.

24. The method of claim 18, wherein the hair is an eyebrow hair and/or an eyelash hair.
